(19) United States
(12) Patent Application Publication
(10) Pub. No.: US 2025/0207193 A1
(43) Pub. Date: Jun. 26, 2025

(54) REUSABLE FLOW CELLS AND METHODS OF USING THEM FOR NUCLEIC ACID SEQUENCING

(71) Applicant: Illumina, Inc., San Diego, CA (US)

(72) Inventors: Jonathan Boutell, Bishops Stortford (GB); Jason Betley, Buntingford (GB); Pietro Gatti Lafranconi, Cambridge (GB); Xiaolin Wu, Cambridge (GB); Oliver Miller, Cambridge (GB)

(73) Assignee: Illumina, Inc., San Diego, CA (US)

(21) Appl. No.: 18/990,018

(22) Filed: Dec. 20, 2024

Related U.S. Application Data
(60) Provisional application No. 63/614,338, filed on Dec. 22, 2023.

(51) Int. Cl. C12Q 1/6874; C12Q 1/6816; C12Q 1/6876

(52) U.S. Cl. CPC C12Q 1/6874; C12Q 1/6816; C12Q 1/6876

(57) ABSTRACT

The present subject matter relates to reusable flow cells, uses of reusable flow cells, methods of manufacturing a reusable flow cell and methods of regenerating a reusable flow cell.

### CROSS-REFERENCE TO RELATED APPLICATIONS

This application claims the benefit of U.S. Provisional Patent Application No. 63/614,338, filed Dec. 22, 2023 and entitled “Reusable Flow Cells and Methods of Using Them for Nucleic Acid Sequencing,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2518-US.xml”, was created on Dec. 18, 2024 and is 13.2 kB in size.

### FIELD

The present application relates to a kit comprising a reusable flow cell, reusable flow cells, and uses of reusable flow cells, methods of manufacturing a reusable flow cell and methods of regenerating a reusable flow cell.

### BACKGROUND

The detection of analytes such as nucleic acid sequences that are present in a biological sample has been used as a method for identifying and classifying microorganisms, diagnosing infectious diseases, detecting and characterizing genetic abnormalities, identifying genetic changes associated with cancer, studying genetic susceptibility to disease, and measuring response to various types of treatment. A common technique for detecting analytes such as nucleic acid sequences in a biological sample is nucleic acid sequencing.

Advances in the study of biological molecules have been led, in part, by improvement in technologies used to characterise the molecules or their biological reactions. In particular, the study of the nucleic acids DNA and RNA has benefited from developing technologies used for sequence analysis.

Methods of nucleic acid amplification which allow amplification products to be immobilised on a solid support in order to form arrays comprised of clusters or “colonies” formed from a plurality of identical immobilised polynucleotide strands and a plurality of identical immobilised complementary strands are known. The nucleic acid molecules present in DNA colonies on the clustered arrays prepared according to these methods can provide templates for sequencing reactions.

One method for sequencing a polynucleotide template involves performing multiple extension reactions using a DNA polymerase to successively incorporate labelled nucleotides to a template strand. In such a “sequencing by synthesis” reaction a new nucleotide strand base-paired to the template strand is built up in the 5′ to 3′ direction by successive incorporation of individual nucleotides complementary to the template strand. In some methods, the step of sequencing a polynucleotide template may be preceded by a template capture step, in which a nucleic acid library is enriched for target nucleic acids before sequencing.

Typically, flow cells utilised in template capture and/or sequencing are used once for a single sequencing run, and are then discarded. There remains a need to develop new flow cells that can be reused for multiple sequencing runs. The present disclosure addresses this need.

### SUMMARY

An aspect of the present disclosure provides a kit comprising:
a. a reusable flow cell, comprising a solid support and linking groups on the solid support surface; wherein the linking groups are configured to:
   i. enable first target-binding oligonucleotides to attach to the solid support via the linking groups; and thereafter
   ii. release the first target-binding oligonucleotides on exposure to a cleaving trigger; and thereafter
   iii. enable second target-binding oligonucleotides to attach to the solid support via the linking groups; and
b. first and second target-binding oligonucleotides.

In an embodiment, the first target-binding oligonucleotides are primers. In an alternative embodiment, the first target-binding oligonucleotides are target capture probes.

In an embodiment, the second target-binding oligonucleotides are primers.

In one embodiment, the first and second target-binding oligonucleotides each comprise one or more linking groups configured to interact with the linking groups on the solid support surface. The linking groups on the first and second target-binding oligonucleotides are as defined herein.

In embodiments, the linking groups are configured to form non-covalent interactions with the first and second target-binding oligonucleotides. In further embodiments the linking groups comprise a biotin moiety or an avidin. In some embodiments the linking groups comprise a biotin.

In an embodiment, the linking groups are configured to form reversible covalent bonds with the first and second target-binding oligonucleotides. In other embodiments the linking groups are configured to form metal-coordination bonds with the first and second target-binding oligonucleotides. Such linking groups may comprise nickel or histidine.

A further aspect of the present disclosure provides a reusable flow cell, comprising a solid support, linking groups on the surface of the solid support and first target-binding oligonucleotides, wherein the first target-binding oligonucleotides are attached to the solid support via the linking groups; and wherein the linking groups are further configured to:
a. release the first target-binding oligonucleotides on exposure to a cleaving trigger; and thereafter
b. enable second target-binding oligonucleotides to attach to the solid support via the linking groups.

In an embodiment, the first target-binding oligonucleotides are primers. In other embodiments, the first target-binding oligonucleotides are target capture probes.

In an embodiment, the second target-binding oligonucleotides are primers.

In embodiments, the linking groups are configured to form non-covalent interactions with the first and second target-binding oligonucleotides. In some embodiments the linking groups comprise a biotin moiety or an avidin, while in some embodiments the linking groups comprise a biotin moiety.

In an embodiment, the linking groups are configured to form reversible covalent bonds with the first and second target-binding oligonucleotides. In other embodiments the linking groups are configured to form metal-coordination bonds with the first and second target-binding oligonucleotides. In some embodiments the linking groups may comprise nickel or histidine.

In some embodiments the linking groups comprise first and second linking groups on the solid support surface; wherein:
a) the first linking group is configured to enable first target-binding oligonucleotides to attach to said solid support via the first linking group;
b) the second linking group is configured to enable second target-binding oligonucleotides to attach to said solid support via the second linking group;
2a) the first linking group is configured to release the first target-binding oligonucleotides on exposure to a first cleaving trigger;
2b) the second linking group is configured to release the second target-binding oligonucleotides on exposure to a second cleaving trigger;
3a) the first linking group is configured to enable new first target-binding oligonucleotides to attach to the solid support via the first linking group; and
3b) the second linking group is configured to enable new second target-binding oligonucleotides to attach to the solid support via the second linking group.

In embodiments, the first linking group is orthogonal to the second linking group.

In some embodiments, the linking groups are attached to the target-binding oligonucleotides. In some embodiments the first linking group is attached to the first target-binding oligonucleotides. In some embodiments the second linking group is attached to the second target-binding oligonucleotides.

In certain embodiments the target-binding oligonucleotides are primers, which may comprise P5 and P7 primers; optionally wherein the P5 primer comprises a sequence comprising SEQ ID NO: 1 or 11 or variant thereof; and/or wherein the P7 primer comprises a sequence comprising SEQ ID NO: 2 or variant thereof.

In some embodiments the target-binding oligonucleotides comprise a biotin moiety or an avidin, and in certain embodiments the target-binding oligonucleotides comprise a biotin moiety. In certain embodiments the linking groups comprise a biotin moiety and the target-binding oligonucleotides comprise a biotin moiety, and the target-binding oligonucleotides are attached to the linking groups by an avidin bridge.

In embodiments the flow cell is configured to allow multiple rounds of release and reattachment of fresh target-binding oligonucleotides.

Further aspects of the present disclosure provide for the use of the reusable flow cell as described herein in sequencing or cluster counting. In embodiments, the use is in pairwise sequencing.

Further aspects of the present disclosure provide a method of manufacturing a reusable flow cell as described herein, said method comprising providing a flow cell, and attaching linking groups, wherein the linking groups are as defined herein.

In an embodiment, the linking groups comprise a biotin moiety and the first and second target-binding oligonucleotides comprise a biotin moiety, and wherein the first and second target-binding oligonucleotides are pre-incubated with an avidin before the step of attaching the target-binding oligonucleotides to the linking groups.

A further aspect of the present disclosure provides a method of regenerating a reusable flow cell, comprising:
providing a reusable flow cell as described herein; wherein the linking groups are attached to first target-binding oligonucleotides;
exposing the reusable flow cell to a cleaving trigger to remove the primers from the linking groups;
attaching second target-binding oligonucleotides to the linking groups.

Further embodiments comprise a method of sequencing or cluster generation comprising providing a reusable flow cell as described herein wherein the linking groups are attached to first target-binding oligonucleotides, exposing the reusable flow cell to a cleaving trigger to remove the primers from the linking groups; and attaching second target-binding oligonucleotides to the linking groups, where the first and second target-binding oligonucleotides are primers.

Further embodiments comprise a method of cluster counting comprising providing a reusable flow cell as described herein wherein the linking groups are attached to first target-binding oligonucleotides, exposing the reusable flow cell to a cleaving trigger to remove the primers from the linking groups; and attaching second target-binding oligonucleotides to the linking groups, where the first and second target-binding oligonucleotides are primers, and wherein the method further comprises counting the clusters.

Further embodiments comprise a method of target capture and optionally target capture and sequencing or cluster generation, comprising providing a reusable flow cell as described herein wherein the linking groups are attached to first target-binding oligonucleotides, exposing the reusable flow cell to a cleaving trigger to remove the primers from the linking groups; and attaching second target-binding oligonucleotides to the linking groups, where the first target-binding oligonucleotides are target capture probes and where the second target-binding oligonucleotides are target capture probes or primers (the second target-binding oligonucleotides are primers where the method is used in target capture and sequencing or cluster generation).

In an embodiment, the cleaving trigger is a thermal trigger, a light trigger or a chemical/biochemical trigger. In embodiments, the cleaving trigger is a solvent wash (for example a hot formamide wash), or is a complexation agent (for example imidazole).

In some embodiments, in which the first and second linkers are orthogonal, the step of exposing the reusable flow cell to the cleaving trigger comprises exposure to a first cleaving trigger to remove the first target-binding oligonucleotide from the first linking group, without removing the second target-binding oligonucleotide from the second linking group. In some embodiments, in which the first and second linkers are orthogonal, the step of exposing the reusable flow cell to the cleaving trigger comprises or further comprises exposure to a second cleaving trigger to remove the second target-binding oligonucleotide from the second linking group, without removing the first target-binding oligonucleotide from the first linking group.

In some embodiments, the method further comprises a step of treating the flow cell with a nuclease prior to the step of attaching the new target-binding oligonucleotide to the linking groups. In some embodiments, the method comprises multiple rounds of release and reattachment of new target-binding oligonucleotides.

### BRIEF DESCRIPTION OF DRAWINGS

FIG. 1 shows a typical template for use in sequencing.

FIG. 2 shows a reusable flow cell based on a streptavidin-biotin P5/P7 surface according to an embodiment provided herein.

FIG. 3 shows a comparison between sequential binding of streptavidin to a flow cell followed by oligos versus preincubation of streptavidin and biotin-P5/P7 and then binding to a flow cell.

FIG. 4 shows that a flow cell having a streptavidin-biotin P5/P7 surface is able to form clusters and undergo sequencing.

FIG. 5 shows the stripping of the streptavidin-biotin P5/P7 surface after sequencing, reloading with fresh streptavidin-biotin P5/P7, and reamplification with a new PhiX library.

FIG. 6 shows the effect of using DNase on run-to-run contamination according to an embodiment provided herein.

FIG. 7 shows the reuse of a flow cell for template capture and enrichment according to an embodiment provided herein.

FIG. 8 shows the reuse of a flow cell for two or more rounds of template capture and enrichment according to an embodiment provided herein.

FIG. 9 shows the use of an enzyme or enzyme complex to add further functionality to a reusable flow cell, according to an embodiment provided herein.

### DETAILED DESCRIPTION

In some examples, the following features apply to all aspects of the present disclosure. In some examples, any suitable combination of the following features apply to all aspects of the present disclosure.

The present disclosure is directed to enabling the reuse of flow cells.

Examples provided in the present disclosure can be used in sequencing, for example pairwise sequencing. Methodology applicable to the present disclosure have been described in WO 08/041002, WO 07/052006, WO 98/44151, WO 00/18957, WO 02/06456, WO 07/107710, WO05/068656, U.S. Ser. No. 13/661,524 and US 2012/0316086, the entire contents of which are herein incorporated by reference. Further information can be found in US 20060024681, US 200602926U, WO 06110855, WO 06135342, WO 03074734, WO07010252, WO 07091077, WO 00179553 and WO 98/44152, the entire contents of which are herein incorporated by reference.

Sequencing generally comprises four fundamental steps: 1) library preparation to form a plurality of template molecules available for sequencing; 2) cluster generation to form an array of amplified single template molecules on a solid support; 3) sequencing the cluster array; and 4) data analysis to determine the target sequence. In addition, where the sequence of the target nucleic acid is known—e.g., the purpose of sequencing is to sequence a specific gene of interest—then an additional step of target enrichment may be performed following library preparation.

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts.” Alternatively, “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size-fractionated prior to modification with the adapter sequences.

As used herein an “adapter” sequence comprises a short sequence-specific oligonucleotide that is ligated to the 5′ and 3′ ends of each DNA (or RNA) fragment in a sequencing library as part of library preparation. The adapter sequence may further comprise non-peptide linkers.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g., linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

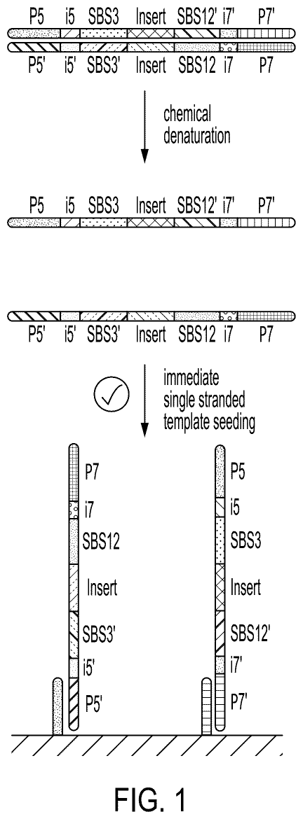

An example of a typical single-stranded nucleic acid template is shown in FIG. 1. In one embodiment, the template comprises, in the 5′ to 3′ direction, a first primer-binding sequence (e.g., P5), an index sequence (e.g., i5), a first sequencing binding site (e.g., SBS3), an insert, a second sequencing binding site (e.g., SBS12), a second index sequence (e.g., i7) and a second primer-binding sequence (e.g., P7′). In another embodiment, the template comprises, in the 3′ to 5′ direction, a first primer-binding site (e.g., P5′, which is complementary to P5), an index sequence (e.g., i5′, which is complementary to i5), a first sequencing binding site (e.g., SBS3′ which is complementary to SBS3), an insert, a second sequencing binding site (e.g., SBS12′, which is complementary to SBS12), a second index sequence (e.g., i7′, which is complementary to i7) and a second primer-binding sequence (e.g., P7, which is complementary to P7′). Either template is referred to herein as a “template strand” or “a single stranded template”. Both template strands annealed together as shown in FIG. 1, is referred to herein as “a double stranded template”. The combination of a primer-binding sequence, an index sequence and a sequencing binding site is referred to herein as an adapter sequence, and a single insert is flanked by a 5′ adapter sequence and a 3′ adapter sequence. The first primer-binding sequence may also comprise a sequencing primer for the index read (i5). “Primer-binding sequences” may also be referred to as “clustering sequences” “clustering primers” “primers” or “cluster primers” in the present disclosure, and such terms may be used interchangeably.

The P5′ and P7′ primer-binding sequences are complementary to short primer sequences (or lawn primers) present on the surface of the flow cells. Binding of P5′ and P7′ to their complements (P5 and P7) on—for example—the surface of the flow cell, permits nucleic acid amplification. As used herein the prime symbol (′) denotes the complementary strand.

The primer-binding sequences in the adapter which permit hybridisation to amplification primers (also referred to herein as flow cell primers) will typically be around 20-40 nucleotides in length, although, in embodiments, the present disclosure is not limited to sequences of this length. The precise identity of the amplification primers, and hence the cognate sequences in the adapters, are generally not material to the present disclosure, as long as the primer-binding sequences are able to interact with amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO05068656, whose contents are incorporated herein by reference in their entirety. The tag can be read at the end of the first read, or equally at the end of the second read, for example using a sequencing primer complementary to the strand marked P7. The present disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221, the entire contents of which are incorporated by reference herein. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicate the starting point of the sequencing read. During the sequencing process, a sequencing primer anneals (i.e., hybridises) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g., SBS3′) followed by synthesis and sequencing of the complementary strand. This leads to the sequencing of the insert. In a second step, an index sequencing primer (e.g., i7 sequencing primer) binds to a second sequencing binding site (e.g., SBS12) leading to synthesis and sequencing of the index sequence (e.g., sequencing of the i7 primer). The second sequencing read may comprise binding of an index sequencing primer (e.g., i5 sequencing primer) to the complement of the first sequencing binding site on the template (e.g., SBS3) and synthesis and sequencing of the index sequence (e.g., i5). In a second step, a second sequencing primer (read 2 sequencing primer) binds to the complement of the primer (e.g., i7 sequencing primer) binds to a second sequencing binding site (e.g., SBS12′) leading to synthesis and sequencing of the insert in the reverse direction.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.). In one embodiment, chemical denaturation is used.

Following denaturation, a single-stranded template library can be contacted in free solution onto a solid support comprising surface capture moieties (for example P5 and P7 primers). This solid support is typically a flow cell, although in alternative embodiments, seeding and clustering can be conducted off-flow cell using, for example, microbeads or the like.

In one embodiment, the sequence of the P5 primer-binding sequence comprises SEQ ID NO: 1 or 11 or a variant thereof, the sequence of the P5′ adapter comprises SEQ ID NO: 3 or 12 or a variant thereof, the sequence of the P7 adapter comprises SEQ ID NO: 2 or a variant thereof and the sequence of the P7′ adapter comprises SEQ ID NO: 4 or a variant thereof.

In embodiments, the variant has at least 80% overall sequence identity to SEQ ID NO: 1, 2, 3 or 4. More preferably, the variant has at least 86%, 87%, 88%, 89%, 90%, 91%, 92%, 93%, 94%, 95%, 96%, 97%, 98%, or at least 99% overall sequence identity to SEQ ID NO: 1, 2, 3 or 4.

The present disclosure is not limited to the primer sequences above. In embodiments, alternative primer sequences can be used.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, TEFLON™, cyclic olefins, polyimides etc.), nylon, ceramics, resins, Zeonor, silica or silica-based materials including silicon and modified silicon, carbon, metals, inorganic glasses, optical fibre bundles, and polymers. A particularly useful material is glass. Other suitable substrate materials may include polymeric materials, plastics, silicon, quartz (fused silica), boro float glass, silica, silica-based materials, carbon, metals including gold, an optical fibre or optical fibre bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g., being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture. Further examples of materials that can be used in the structured substrates or methods of the present disclosure are described in U.S. Ser. No. 13/661,524 and US Pat. App. Pub. No. 2012/0316086 A1, the entire contents of each of which are incorporated herein by reference.

The present disclosure may make use of solid supports comprised of a substrate or matrix (e.g., glass slides, polymer beads etc) which has been “functionalised”, for example by application of a layer or coating of an intermediate material comprising reactive groups which permit reversible covalent attachment to biomolecules, such as polynucleotides. Examples of such supports include, but are not limited to, a substrate such as glass. Alternatively, the substrate such as glass may be treated to permit direct reversible covalent attachment of a biomolecule; for example, glass may be treated with hydrochloric acid, thus exposing the hydroxyl groups of the glass, and phosphite-triester chemistry used to directly attach a nucleotide to the glass via a reversible covalent bond between the hydroxyl group of the glass and the phosphate group of the nucleotide.

In other embodiments, the solid support may be “functionalised” by application of a layer or coating of an intermediate material comprising groups that permit non-covalent attachment to biomolecules. In such embodiments, the groups on the solid support may form one or more of ionic bonds, hydrogen bonds, hydrophobic interactions, 71-71 interactions, van der Waals interactions and host-guest interactions, to a corresponding group on the biomolecules (e.g., polynucleotides). The interactions formed between the group on the solid support and the corresponding group on the biomolecules may be configured to cause immobilisation or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

In other embodiments, the solid support may be “functionalised” by application of an intermediate material comprising groups that permit attachment via metal-coordination bonds to biomolecules. In such embodiments, the groups on the solid support may include ligands (e.g., metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilisation or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilisation or attachment of molecules (e.g., nucleic acids) to a solid support, the terms “immobilised” and “attached” are used interchangeably herein and both terms are intended to encompass direct or indirect, reversible covalent or non-covalent attachment, metal-coordination attachment, unless indicated otherwise, either explicitly or by context. In certain embodiments provided herein, reversible covalent attachment may be preferred; in other embodiments, attachment using non-covalent interactions may be preferred; in yet other embodiments, attachment using metal-coordination bonds may be preferred. However, in general the molecules (e.g., nucleic acids) remain immobilised or attached to the support under the conditions in which it is intended to use the support, for example in applications requiring nucleic acid amplification and/or sequencing. When referring to attachment of nucleic acids to other nucleic acids, then the terms “immobilised” and “hybridised” are used herein, and generally refer to hydrogen bonding between complementary nucleic acids.

If the amplification is performed on beads, either with a single or multiple extendable primers, the beads may be analysed in solution, in individual wells of a microtitre or picotitre plate, immobilised in individual wells, for example in a fibre optic type device, or immobilised as an array on a solid support. The solid support may be a planar surface, for example a microscope slide, wherein the beads are deposited randomly and held in place with a film of polymer, for example agarose or acrylamide.

As described above, once a library comprising template nucleotide strands has been prepared, the templates are seeded onto a solid support and then amplified to generate a cluster of single template molecules.

By way of brief example, following attachment of the P5 and P7 primers, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e., either P5′ or P7′) which is capable of bridging to the second primer molecule immobilised on the solid support and binding. Further rounds of amplification (analogous to a standard PCR reaction) lead to the formation of clusters or colonies of template molecules bound to the solid support.

Thus, solid-phase amplification by either the method analogous to that of WO 98/44151 or that of WO 00/18957 (the contents of which are incorporated herein in their entirety by reference) will result in production of a clustered array comprised of colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582. Further information on amplification can be found in WO0206456 and WO07107710, the contents of which are incorporated herein in their entirety by reference. Through such approaches, a cluster of single template molecules is formed.

To facilitate sequencing, it is preferable if one of the strands is removed from the surface to allow efficient hybridisation of a sequencing primer to the remaining immobilised strand. Suitable methods for linearisation are described in more detail in application number WO07010251, the contents of which are incorporated herein by reference in their entirety.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. In a particular embodiment, the label is a fluorescent label. Each nucleotide type may carry a different fluorescent label. However, the detectable label need not be a fluorescent label. Any label can be used which allows the detection of the incorporation of the nucleotide into the DNA sequence. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

Alternative methods of sequencing include sequencing by ligation, for example as described in U.S. Pat. No. 6,306,597 or WO06084132, the entire contents of which are incorporated herein by reference.

In some embodiments, sequencing may involve pairwise sequencing. The typical steps of pairwise sequencing are known and have been described in WO 2008/041002, the entire contents of which are herein incorporated by reference. However, the key steps will be briefly described.

Pairwise sequencing relates to methods for sequencing two regions of a target double-stranded polynucleotide template, referred to herein as the first and second regions for sequence determination. The first and second regions for sequence determination are at both ends of complementary strands of the double-stranded polynucleotide template, which are referred to herein respectively as first and second template strands. Once the sequence of a strand is known, the sequence of its complementary strand is also known, therefore the term two regions can apply equally to both ends of a single stranded template, or both ends of a double stranded template, wherein a first region and its complement are known, and a second region and its complement are known.

A plurality of template polynucleotide duplexes are immobilised on a solid support. The template polynucleotides may be immobilised in the form of an array of amplified single template molecules, or ‘clusters’. Each of the duplexes within a particular cluster comprises the same double-stranded target region to be sequenced. The duplexes are each formed from complementary first and second template strands which are linked to the solid support at or near to their 5′ ends. Typically, the template polynucleotide duplexes will be provided in the form of a clustered array.

An alternate starting point is a plurality of single stranded templates which are attached to the same surface as a plurality of primers that are complementary to the 3′ end of the immobilised template. The primers may be reversibly blocked to prevent extension. The single stranded templates may be sequenced using a hybridised primer at the 3′ end. The sequencing primer may be removed after sequencing, and the immobilised primers deblocked to release an extendable 3′ hydroxyl. These primers may be used to copy the template using bridged strand resynthesis to produce a second immobilised template that is complementary to the first. Removal of the first template from the surface allows the newly single stranded second template to be sequenced, again from the 3′ end. Thus, both ends of the original immobilised template can be sequenced. Such a technique allows paired end reads where the templates are amplified using a single extendable immobilised primer, for example as described in the following references, the entire contents of each of which are incorporated by reference herein: Mitra et al., “In situ localized amplification and contact replication of many individual DNA Molecules,” Nucleic Acids Research 27(24) e34: pp. 1-6 (1999); Shendure et al., “Accurate Multiplex Polony Sequencing of an Evolved Bacterial Genome,” Science 309(5741): pp. 1728-1732 (2005); and Margulies et al., “Genome Sequencing in microfabricated high-density picolitre reactors,” Nature 437: pp. 376-380 (2005).

In some examples, by controlling the nature of the immobilisation or attachment of target-binding oligonucleotides to the solid support, it is possible to provide reusable flow cells. While the present subject matter is described in relation to target-capture probes and primers, in embodiments, other biomolecules may be immobilised/attached to the solid substrate and removed and reapplied to make the flow cell reusable. The primers may contain additional components, for example linker groups or other groups to facilitate function.

The reusable flow cells may, for example, be used in sequencing (e.g., pairwise sequencing).

The reusable flow cells may, for example, be used in cluster counting. In such embodiments, the flow cell may be amplified as described above, stained (e.g., with fluorescent dyes) and then counted (e.g., using detection means as described herein).

The reusable flow cell may also be used, for example, in target capture, optionally followed by the use of the reusable flow cell in sequencing or cluster generation or cluster counting.

The reusable flow cell may also be used in methods of sequencing, cluster generation, cluster counting and/or target capture, as described herein.

Therefore, some examples provided herein are directed to a reusable flow cell comprising a solid support and linking groups on the solid support surface; wherein the linking groups are configured to:
1) enable first target-binding oligonucleotides to attach to the solid support via the linking groups; thereafter
2) release the first target-binding oligonucleotides on exposure to a cleaving trigger; and thereafter
3) enable second target-binding oligonucleotides to attach to the solid support via the linking groups.

Some examples herein are directed to a kit comprising:
a. a reusable flow cell, comprising a solid support and linking groups on the solid support surface; wherein the linking groups are configured to:
   i. enable first target-binding oligonucleotides to attach to the solid support via the linking groups; and thereafter
   ii. release the first target-binding oligonucleotides on exposure to a cleaving trigger; and thereafter
   iii. enable second target-binding oligonucleotides to attach to the solid support via the linking groups; and
b. first and second target-binding oligonucleotides.

In one embodiment, the kit further comprises a transposase or transposome complex, for example as described herein. In an alternative embodiment, the kit does not comprise a transposase or transposome complex.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxy cytidine diphosphate (dCDP), deoxycytidine triphosphate (dCTP), deoxyguanosine monophosphate (dGMP), deoxy guanosine diphosphate (dGDP), deoxy guanosine triphosphate (dGTP), deoxyuridine monophosphate (dUMP), deoxyuridine diphosphate (dUDP), and deoxyuridine triphosphate (dUTP). The term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates

By “target-binding oligonucleotide” is meant any oligonucleotide that binds to a target nucleic acid or a portion of thereof. The target sequence may be considered to be the whole sequence that is desired to be identified. However, a “target-binding oligonucleotide” binds only to a portion (i.e. not the whole) of the target sequence. In some embodiments, the target-binding oligonucleotides are no more than 150 nucleotides long, more often between 18 and 25 or between 80 and 120 nucleotides long. Where the target-binding oligonucleotide is a primer, the target-binding oligonucleotide may be between 18 and 25 nucleotides long. Where the target-binding oligonucleotides is a target-capture probe, the target-binding oligonucleotides may be between 80 and 120 nucleotides long. In some embodiments, the target-binding oligonucleotide comprises a portion of the complement of the target sequence. The target nucleic acid may be DNA or RNA. The sequence of the target nucleic acid is not limited and is immaterial for the purpose of the present disclosure.

As used herein, “binds” and “hybridises” can be used interchangeably. The skilled person would understand that the term “hybridize” is intended to mean non-covalently associating a first polynucleotide strand to a second polynucleotide strand. In one instance, a first single-stranded polynucleotide may non-covalently bind to a second single-stranded polynucleotide through complimentary base pairing to form a double-stranded duplex. By “duplex” is meant a double-stranded polynucleotide. This process, of a first polynucleotide binding to a second polynucleotide, is called “hybridisation”.

For hybridisation to occur, the first polynucleotide and the second polynucleotide must be “substantially complementary” to one another. That is, the first and the second polynucleotide must have a sufficient proportion of complementary bases to one another to allow complementary base pairing, or Watson-Crick base pairing, to occur and associate the two polynucleotide strands. The proportion of complementary bases required for two polynucleotide strands to be “substantially complementary”, and therefore for hybridisation to occur, will vary according to the nucleotide sequence of the first and the second polynucleotide strands and the conditions of hybridisation. Accordingly, hybridisation may occur along part or the whole of each of the first and second polynucleotide strand in the duplex. Similarly, the site of hybridisation along each of the first and second polynucleotide strands will depend on the degree of complementarity between the nucleotide sequences along each of the first and the second polynucleotide strands.

The strength of the association between the hybridised first polynucleotide and second polynucleotide increases with the complementarity between the nucleotide sequences of the first and the second polynucleotide strands, i.e., the degree of homology between the first and the second polynucleotide strands. The strength of hybridization between a first and a second polynucleotide strand may be characterized by a temperature of melting (Tm) at which 50% of the duplexes disassociate from one another.

The conditions of hybridisation, hereafter referred to as the “stringency”, will alter the frequency of hybridisation between a first and a second polynucleotide. By “high stringency conditions” is meant conditions, which include high hybridization temperatures and a low concentration of salt in buffers, that promote the hybridisation of highly homologous nucleic acid sequences. By “low stringency conditions” is meant conditions, which include low hybridization temperatures and a high concentration of salt in buffers, that promote the hybridization of less homologous nucleic acid sequences. When the conditions of stringency are too low, probes bind to unrelated targets. The use of target capture probes is useful for targeted sequencing. Typically to perform targeted sequencing, the library is first enriched for the sequence of interest. This may be referred to as target capture or library enrichment (such terms may be used interchangeably). The target capture probe may be of one or more different sequence. That is, multiple different target capture probes corresponding to multiple different target sequences may be used. Alternatively, just one type (sequence) of target capture probe may be used, meaning that just one target nucleic acid is captured and enriched.

In the present disclosure, the flow cell is used for library enrichment. Specifically, the flow cell is a reusable flow cell, as described herein. Initially the flow cell is immobilised with a plurality of target-binding oligonucleotides. In this embodiment, all of the plurality of target-binding oligonucleotides are target capture probes. Once target capture is complete (e.g., the library is enriched for the target nucleic acid), the library may be eluted from the flow cell and collected. The flow cell can then be stripped of the capture probes and primer sequences reattached. Accordingly, in one embodiment, the second target-binding oligonucleotides are primers.

The collected library can then be flowed back over the flow cell for cluster amplification and sequencing. As such, using a single flow cell a library can be firstly enriched for a target nucleic acid before the target nucleic acid is subsequently sequenced. That is, the flow cell is reused.

As provided herein, using the reusable flow cell as a vehicle for library enrichment prior to sequencing has a number of advantages. Firstly, because the flow cell can be reused, in the first step, all immobilised target-binding oligonucleotides may be target capture probes. That is, it is unnecessary for the flow cell to have a mixture of flow cell primers and capture probes—since in later steps the capture probes can be removed and replaced with the flow cell primers. This, in turn not only improves the efficiency of target capture (i.e., because there are more capture probes on the flow cell), but also the time taken to perform target capture (again, because there are more capture probes). Secondly, the presence of target capture probes on a solid support could give rise to non-specific amplification during subsequent clustering steps. The complete removal of target capture probes prior to sequencing negates this possible issue.

In addition to the above advantages, the reusable flow cell provided herein may be used to perform multiple rounds of enrichments before sequencing. Accordingly, in one embodiment, the linking groups are configured to enable third, fourth and so on target-binding oligonucleotides to attach to the solid support by the linking groups.

First, second, third and so on probes may differ in sequence—for example, they may target different regions of the target nucleic acid. Alternatively, the sequence of the target capture probes may, with each round, become increasingly more specific (i.e., the level or percentage of complementarity may increase) to the target nucleic acid.

In an alternative embodiment, the first and second target-binding oligonucleotides are primers.

As used herein, the term “primer” refers to a polynucleotide to which nucleotides may be added via a free 3′ OH group. The primer length may be any suitable number of bases long and may include any suitable combination of natural and non-natural nucleotides. Typically, primers are approximately 18 to 25 bp in length.

A target nucleic acid may include an “adapter” that hybridizes to (has a sequence that is complementary or substantially complementary to) a primer, and may be amplified so as to generate a complementary copy polynucleotide by adding nucleotides to the free 3′ OH group of the primer. A “flow cell primer” is intended to mean a primer that may hybridize to a first adapter of a target polynucleotide. The first adapter may have a sequence that is complementary to that of the flow cell primer.

The primers may comprise first primers (e.g., P5 primers) and second primers (e.g. P7 primers) and both primers can attach to the solid support via the linking groups. That is, the first target-binding oligonucleotides may comprise first and second primers and the second target-binding oligonucleotides may comprise first and second primers (e.g. P5 and P7 primers respectively). Where both the first and second target-binding oligonucleotides are primers, the second target-binding oligonucleotides may be referred to as new primers.

By new primers it is intended to mean that fresh unused primers can be applied to the flow cell following removal of the previous primers. It is therefore possible to use a flow cell in a sequencing analysis, strip the flow cell to remove the prior material and reapply fresh primers which allow the flow cell to be reused.

In one embodiment, the target-binding oligonucleotide(s) may additionally be used to localise an enzyme or enzyme complex to a relevant site of the flow cell. The localisation of an enzyme or enzyme complex to a flow cell, or region thereof, confers the benefit of additional functionality. By “enzyme” or “enzyme complex” is meant any biological structure that may catalyse a reaction.

In one example, the target-binding oligonucleotide(s) may bind to an enzyme or enzyme complex by hybridising to a sequence attached to or forming part of an enzyme or enzyme complex. For example, a or a portion of an immobilised target-binding oligonucleotide(s) may hybridise to a sequence that is bound to an enzyme, thereby localising the enzyme to a location on the flow cell.

In an alternative example, the target-binding oligonucleotide(s) may comprise (i.e., may be a part of) an enzyme or enzyme complex directly (e.g., may be covalently linked to). In other words, the first or second target-binding oligonucleotide(s) may be bound or linked to an enzyme or enzyme complex at the time it is flowed onto a solid surface.

The method of association between a target-binding oligonucleotide(s) and an enzyme or enzyme complex is immaterial to the present disclosure; the principle is that an enzyme or enzyme complex localised to the target-binding oligonucleotides may confer additional functionality to the reusable flow cell. Certainly, the skilled person can readily devise a plethora of appropriate methods of linking an enzyme to the reusable flow cell.

In an embodiment, the enzyme or an enzyme complex comprises or consists of a transposase, optionally as part of a transposome complex. However, any enzyme capable of fragmenting and tagging a nucleic acid as described herein, such as an integrase, may also be used.

By “transposase” is meant an enzyme that binds a transposon end sequence to form a functional complex that may catalyse a transposition reaction. A transposition reaction is a reaction wherein one or more transposons are inserted into target nucleic acids at random sites or almost random sites. By “transposon” is meant a polynucleotide sequence including a sequence that may be inserted into a (different) target polynucleotide by a transposase. For example, a transposase may insert a transposon comprising (for example an adapter and an index sequence into a target sequence after the target sequence binds to the transposase via its transposon end sequence. By

“transposon end sequence” is meant the sequence of DNA that a transposase recognizes and binds to. The transposon end sequence flanks the transposon, also known as a “transposon element”, which is the DNA fragment that is transposed during a transposition reaction.

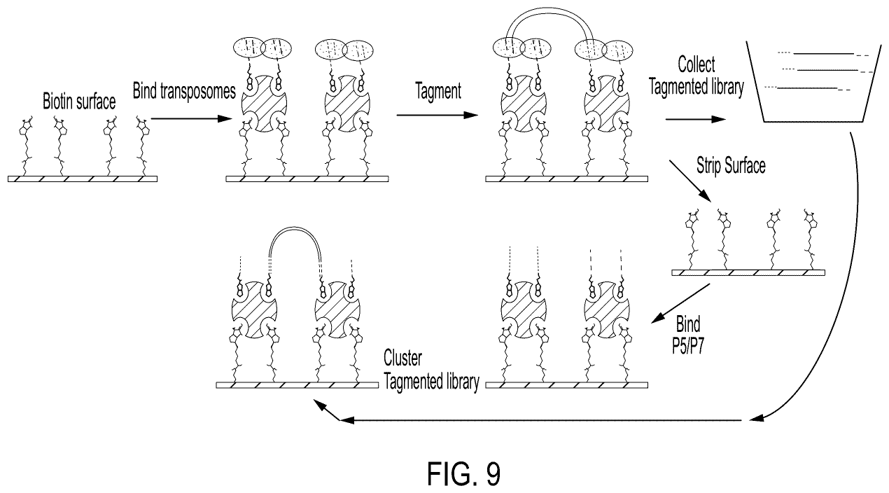

Accordingly, in one embodiment, the first target-binding polynucleotide is a transposon that may be hybridised to a target sequence to form a library strand (see, for example, FIG. 9).

In some aspects, the transposon end sequence is a double-stranded transposon end sequence. The transposase binds to a transposase end site in a target nucleic acid and inserts the transposon into a target nucleic acid. In some such insertion events, one strand of the transposon end sequence (or recognition sequence) is transferred into the target nucleic acid, resulting in a cleavage event.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Tyl, Tn7 transposase, Tn/O and IS 10, Mariner transposase, Tel, P Element, Tn3, bacterial insertion sequences, retroviruses, and retrotransposon of yeast. More examples include IS5, TnlO, Tn903, IS 911, and engineered versions of transposase family enzymes. The embodiments described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, the entire contents of which are incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998, the entire contents of which are incorporated by reference herein). In one embodiment, a transposase recognition site that forms a complex with a hyperactive Tn5 transposase is used (e.g., EZ-Tn5™ Transposase, Epicentre Biotechnologies, Madison, Wis.). In some embodiments, the Tn5 transposase is a wild-type Tn5 transposase. In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adapter sequence in each monomer and the second population has a different adapter sequence in each monomer.

A “transposome complex” is comprised of at least one transposase (or other enzyme as described herein) and a transposon recognition sequence. However, a transposome complex may additionally comprise some or all of the components of a transposon recognition reaction described herein. A transposome complex can simultaneously fragment and tag (“tagmentation”) nucleic acids bound at their transposon end sequence, thereby creating a population of fragmented nucleic acid molecules. In some embodiments, adapter sequences are transferred to the nucleic acid fragment by a tagmentation reaction. The adapter sequence can include one or more functional sequences or components (e.g., primer sequences, anchor sequences, universal sequences, spacer regions, or index tag sequences) as needed or desired.

In an exemplary embodiment of a transposome complex, the transposome complex comprises a transposase, a first transposon comprising a 3′ transposon end sequence and a 5′ adapter sequence; and a second transposon comprising a 5′ transposon end sequence complementary to at least a portion of the 3′ transposon end sequence and a 3′ adapter sequence.

In any of the embodiments described herein, the first transposon may include a 5′ adapter sequence and the second transposon may include a 3′ adapter sequence. Adapter sequences may comprise one or more functional sequences or components selected from the group consisting of primer sequences, anchor sequences, universal sequences, spacer regions, index sequences, capture sequences, barcode sequences, cleavage sequences, sequencing-related sequences, and combinations thereof. In some embodiments, an adapter sequence comprises a primer sequence. In other embodiments, an adapter sequence comprises a primer sequence and an index or barcode sequence. A primer sequence may also be a universal sequence. This disclosure is not limited to the type of adapter sequences that could be used and a skilled artisan will recognize additional sequences that may be of use for library preparation and next generation sequencing. A universal sequence is a region of nucleotide sequence that is common to two or more nucleic acid fragments. Optionally, the two or more nucleic acid fragments also have regions of sequence differences. A universal sequence that may be present in different members of a plurality of nucleic acid fragments can allow for the replication or amplification of multiple different sequences using a single universal primer that is complementary to the universal sequence.

In any of the embodiments described herein, the first transposon may include a 3′ transposon end sequence and the second transposon may include a 5′ transposon end sequence. In some embodiments, the 5′ transposon end sequence is at least partially complementary to the 3′ transposon end sequence. In some embodiments, the complementary transposon end sequences hybridize to form a double-stranded transposon end sequence that binds to the transposase (or other enzyme as described herein). In some embodiments, the transposon end sequence is a mosaic end (ME) sequence. Thus, in some embodiments, the 3′ transposon end sequence is an ME sequence and the 5′ transposon end sequence is an ME′ sequence.

In any of the embodiments, the adapter sequence or transposon end sequences may be defined as SEQ ID NO: 5, 6, 7, 8, 9 or 10 or a variant thereof. In embodiments, the variant has at least 80% overall sequence identity to SEQ ID NO: 5, 6, 7, 8, 9 or 10. More preferably, the variant has at least 86%, 87%, 88%, 89%, 90%, 91%, 9%, 93%, 94%, 95%, 96%, 97%, 98%, or at least 99% overall sequence identity to SEQ ID NO: 5, 6, 7, 8, 9 or 10.

For example, in one embodiment provided herein, a first target-binding oligonucleotide comprises a sequence that facilitates binding to a transposome complex comprising a transposase, and a first transposon comprising a 3′ transposon end sequence and a 5′ adapter sequence. Alternatively, the first target-binding oligonucleotide comprises the above-described transposome complex (i.e., forms part of the complex). Similarly, a (different) first target-binding oligonucleotide(s) comprises sequence that facilitates binding to or (itself) comprises of a transposome complex comprising a transposase and a second transposon comprising a 5′ transposon end sequence complementary to at least a portion of the 3′ transposon end sequence and a 3′ adapter sequence.

When DNA is flowed over the flow cell, each of the transposome complexes may bind to the 3′ or 5′ transposon end sequence of the polynucleotide respectively, capturing it onto the flow cell. As such, duplex nucleotide sequences may be captured on the flow cell by binding to the target-recognition sequence of spatially resolved transposome complexes.

The transposome complex can simultaneously fragment and tag (“tagmentation”) the captured sequence, thereby creating a population of fragmented nucleic acid molecules tagged with unique adapter sequences at the ends of the fragments. The simultaneous fragmentation of the DNA and ligation of adapters to the 5′ ends of both strands of the duplex allows dual-indexed paired-end libraries to be prepared from a DNA sample in a much reduced time-frame relative to conventional methods. Further, by configuring the distribution of streptavidin-bio-target capture probe-transposome complexes it is possible to capture a library strand of a desired insert length, thereby increasing the efficiency of subsequent sequencing.

In one embodiment, the first and second target-binding oligonucleotides also each comprise one or more linking groups configured to interact with the linking groups on the solid support surface. The linking groups on the first and second target-binding oligonucleotides are as defined below. The linking groups on each of the first and second target-binding oligonucleotides may however be biotin.

In any of the above-described aspects, the linking groups may be configured to form non-covalent interactions, reversible covalent bonds, or metal-coordination bonds with the target-binding oligonucleotides.

Advantageously, this strategy does not require complex enzymatic manipulation steps, which may not be as efficient as desired, and the primers being used each time are “fresh”. As such, this strategy can advantageously be used to re-use the flow cells with different sequences of surface oligos from run to run. In some embodiments, the target-binding oligonucleotides may be attachable to the linking groups on the solid support by non-covalent interactions. These non-covalent interactions may include one or more of ionic bonds, hydrogen bonds, hydrophobic interactions, π-π interactions, van der Waals interactions and host-guest interactions. Where non-covalent interactions are used, the type of interaction is not particularly limited, provided that the interactions are (collectively) sufficiently strong for the target-binding oligonucleotides to remain attached to the solid support during target capture or template extension. The non-covalent interactions may also be weak enough such that the target-binding oligonucleotides can then be removed from the solid support on exposure to a cleavage trigger.

Preferably, the non-covalent interaction is one formed between an avidin (e.g., streptavidin) and biotin. In some embodiments, both the solid support and the target-binding oligonucleotides may comprise biotin, and the target-binding oligonucleotides may attach to the solid support via an avidin (e.g., streptavidin) bridging intermediary. In other embodiments, the solid support may comprise biotin, and attachable to an avidin (e.g., streptavidin) on the target-binding oligonucleotides. In other embodiments, the solid support may comprise an avidin (e.g., streptavidin), and attachable to a biotin moiety on the target-binding oligonucleotides.

As used herein, the term “ionic bond” refers to a chemical bond between two or more ions that involves an electrostatic attraction between a cation and an anion. For example, the cation may be selected from “metal cations”, as described herein, or “non-metal cations”. Non-metal cations may include ammonium salts (e.g., alkylammonium salts) or phosphonium salts (e.g. alkylphosphonium salts). The anion may be selected from phosphates, thiophosphates, phosphonates, thiophosphonates, phosphinates, thiophosphinates, sulfates, sulfonates, sulfites, sulfinates, carbonates, carboxylates, alkoxides, phenolates and thiophenolates.

As used herein, the term “hydrogen bond” refers to a bonding interaction between a lone pair on an electron-rich atom (e.g., nitrogen, oxygen or fluorine) and a hydrogen atom attached to an electronegative atom (e.g., nitrogen or oxygen).

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

In other embodiments, the target-binding oligonucleotides may be attachable to the linking groups on the solid support by covalent bonds. Where covalent bonds are used, the bond may be stable such that target-binding oligonucleotides remain attached to the solid support during amplification and/or sequencing. The covalent bond may be a reversible covalent bond so that the target-binding oligonucleotides can be removed from the solid support on exposure to a cleavage trigger.

As used herein, the term “reversible covalent bond” refers to a covalent bond that can be cleaved for example under the application of heat, light or other (bio)chemical methods (e.g., by exposure to a degradation agent, such as an enzyme or a catalyst), while a “non-reversible covalent bond” is stable to degradation under such conditions. Non-limiting examples of reversible covalent bonds include thermally or photolytically cleavable cycloadducts (e.g., furan-maleimide cycloadducts), alkenylene linkages, esters, amides, acetals, hemiaminal ethers, aminals, imines, hydrazones, polysulfide linkages (e.g., disulfide linkages), boron-based linkages (e.g., boronic and borinic acids/esters), silicon-based linkages (e.g., silyl ether, siloxane), and phosphorus-based linkages (e.g., phosphite, phosphate) linkages.

As used herein, the term “cycloadduct” refers to a cyclic structure formed from a cycloaddition reaction between two components (e.g., Diels-Alder type cycloaddition between a diene and a dienophile, or 1,3-dipolar type cycloaddition between a dipole and a dipolarophile). The “cycloadduct” may be cleavable and undergo a retro-cycloaddition reaction to regenerate the two components (e.g., thermally or photolytically).

As used herein, the term “alkyl” refers to monovalent straight and branched chain groups respectively having from 1 to 12 carbon atoms. Preferably, the alkyl groups are straight or branched alkyl groups having from 1 to 6 carbon atoms, more preferably straight or branched alkyl groups having from 1 to 4 carbon atoms. An alkyl group may comprise one or more “substituents”, as described herein.

As used herein, the term “alkenyl” or “alkenylene” refers to monovalent or divalent straight and branched chain groups respectively having from 1 to 12 carbon atoms, and which comprise at least one carbon-carbon double bond. Preferably, the alkenyl or alkenylene groups are straight or branched alkenyl or alkenylene groups having from 1 to 6 carbon atoms, more preferably straight or branched alkenyl or alkenylene groups having from 1 to 4 carbon atoms. An alkenyl or alkenylene group may comprise one or more “substituents”, as described herein.

As used herein, the term “alkynyl” refers to monovalent straight and branched chain groups respectively having from 1 to 12 carbon atoms, and which comprise at least one carbon-carbon triple bond. Preferably, the alkynyl groups are straight or branched alkynyl groups having from 1 to 6 carbon atoms, more preferably straight or branched alkynyl groups having from 1 to 4 carbon atoms. An alkynyl group may comprise one or more “substituents”, as described herein.

As used herein, the term “amino” refers to a —N(R)(R′) group, where R and R′ are independently hydrogen or a “substituent” as defined herein. As used herein, the term “amine linkage” refers to a —NR— group, and where R is hydrogen or a “substituent” as defined herein.

As used herein, the term “ester” refers to a —O—C(═O)— group, where the group is attached to two other carbon atoms at the points of attachment to the group.

As used herein, the term “amide” refers to a —NR—C(═O)— group, where R is hydrogen or a “substituent” as described herein.

An “aryl” group refers to a monovalent monocyclic, bicyclic or tricyclic aromatic group respectively containing from 6 to 14 carbon atoms in the ring. Common aryl groups include C₆-C₁₄ aryl, for example, C₆-C₁₀ aryl. An aryl group may comprise one or more “substituents”, as described herein.

A “heterocycloalkyl” group refers to a monovalent saturated or partially saturated 3 to 7 membered monocyclic, or 7 to 10 membered bicyclic ring system respectively, which consists of carbon atoms and from one to four heteroatoms independently selected from the group consisting of O, N, and S, wherein the nitrogen and sulfur heteroatoms may be optionally oxidised, the nitrogen may be optionally quaternised, and includes any bicyclic group in which any of the above-defined rings is fused to a benzene ring, and wherein the ring may be substituted on carbon or on a nitrogen atom if the resulting compound is stable. Non-limiting examples of “heterocycloalkyl” groups include pyrrolidinyl, tetrahydrofuranyl, dihydrofuranyl, tetrahydrothienyl, tetrahydrothiopyranyl, isoxazolinyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydropyridazinyl (e.g. 1,4-dihydropyridazinyl), pyrazolidinyl, imidazolinyl, imidazolidinyl, 3-azabicyclo[3.1.0]hexyl, 3-azabicyclo[4.1.0]heptyl, 3H-indolyl, and quinolizinyl. A heterocycloalkyl group may comprise one or more “substituents”, as described herein.

A “heteroaryl” group refers to monovalent aromatic groups having 5 to 14 ring atoms respectively (for example, 5 to 10 ring atoms) and containing carbon atoms and 1, 2 or 3 oxygen, nitrogen or sulfur heteroatoms. Non-limiting examples of “heteroaryl” groups include quinolyl including 8-quinolyl, isoquinolyl, coumarinyl including 8-coumarinyl, pyridyl, pyrazinyl, pyrazolyl, pyrimidinyl, pyridazinyl, furyl, pyrrolyl, thienyl, thiazolyl, isothiazolyl, triazolyl (e.g. 1,2,3-triazolyl), tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanylene, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl and furopyridyl. Where the heteroaryl (or heteroarylene) group contains a nitrogen atom in a ring, such nitrogen atom may be in the form of an N-oxide, e.g., a pyridyl N-oxide, pyrazinyl N-oxide, pyrimidinyl N-oxide and pyridazinyl N-oxide. A heteroaryl group may comprise one or more “substituents”, as described herein.

As used herein, the term “ester” refers to a —O—C(═O)— group, where the group is attached to two other carbon atoms at the points of attachment to the group.

As used herein, the term “amide” refers to a —NR—C(═O)— group, where R is hydrogen or a “substituent” as described herein.

As used herein, the term “acetal” refers to a —OC(R)(R′)O— group, where R and R′ are independently hydrogen or a “substituent” as described herein.

As used herein, the term “hemiaminal ether” refers to a —OC(R)(R′)NR″— group, where R, R′ and R″ are independently hydrogen or a “substituent” as described herein.

As used herein, the term “aminal” refers to a —NR(R′)(R″)NR′″— group, where R, R′, R″ and R′″ are independently hydrogen or a “substituent” as described herein.

As used herein, the term “imine” refers to a —C(R)═N— group, where R is hydrogen or a “substituent” as described herein.

As used herein, the term “hydrazone” refers to a —C(R)═N—NR′— group, where R and R′ are independently hydrogen or a “substituent” as described herein.

As used herein, the term “polysulfide” refers to a —(S)ₙ— group, wherein n is 2 to 10, or 2 to 6. For example, n may be 2, forming a “disulfide” linkage.

As used herein, the term “boron-based linkage” refers to a —(O)ₐ—B(OR)—(O)_{b}— group, where R is independently hydrogen or a “substituent” as described herein, and where a and b are independently 0 or 1.

As used herein, the term “silicon-based linkage” refers to a —(O)ₐ—Si(R)(R′)—(O)_{b}— group, where R and R′ are independently hydrogen or a “substituent” as described herein, and where a and b are independently 0 or 1.

As used herein, the term “phosphorus-based linkage” refers to a —(O)ₐ—P(R)—(O)_{b}— group, where R and R′ are independently hydrogen or a “substituent” as described herein, and where a and b are independently 0 or 1.

As used herein, the term “substituent” refers to groups such as OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, protected OH, protected amino, protected SH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heterocycloalkyl, and substituted or unsubstituted heteroaryl, where each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, substituted or unsubstituted heterocycloalkyl, and substituted or unsubstituted heteroaryl. Where such groups are themselves substituted, the substituents may be chosen from the foregoing list. In addition, where there are more than one R′ groups on a substituent, each R′ may be the same or different.

In other embodiments, the target-binding oligonucleotides may be attachable to the linking groups on the solid support by metal-coordination bonds. Where metal-coordination bonds are used, the bond may be strong enough such that the target-binding oligonucleotides remain attached to the solid support during amplification and/or sequencing. The metal-coordination bond may be reversibly formed so that the target-binding oligonucleotides can be removed from the solid support on exposure to a cleavage trigger.

Preferably, the metal-coordination bond is one formed between nickel and histidine, such as nickel-His6 tag. The solid support may comprise nickel (e.g., nickel metal or nickel ions), and attachable to a histidine (e.g., His6 tag) moiety on the target-binding oligonucleotides. Alternatively, the solid support may comprise a histidine (e.g., His6 tag), and attachable to nickel (e.g., nickel metal or nickel ions) on the target-binding oligonucleotides.

As used herein, the term “metal-coordination bond” refers to a reversible ionic bond and/or a reversible dative covalent bond formed between a metal moiety and a ligand (e.g. a “metal-coordination group”, as described herein).

As used herein, the term “metal-coordination group” refers to a group which is able to coordinate with a metal moiety by forming a reversible ionic bond and/or a reversible dative covalent bond between the coordinating group and the metal moiety. Non-limiting examples of metal-coordination groups include benzenediols (e.g., catechols) or derivatives thereof; benzenetriols (e.g., gallols) or derivatives thereof; amino acids including histidine (e.g., polyhistidines such as His6 tag), serine, threonine, asparagine, glutamine, lysine, or cysteine; and ethylenediaminetetraacetic acid and derivatives thereof.

The ratio of metal-coordination group(s) to metal moieties can be tuned. There may be one, two or three coordinating groups per metal moiety.

As used herein, a “metal moiety” can be any metal moiety suitable to form ionic bonds, or to coordinate with a metal-coordinating group. For the metal-coordinating group, the metal moiety forms reversible ionic bonds and/or reversible dative covalent bonds with metal-coordination group(s). Suitable metal moieties include metal cations, metal oxides, metal hydroxides, metal carbides, metal nitrides and/or metal nanoparticles.

Particular metal cations include lithium, sodium, potassium, rubidium, caesium, beryllium, magnesium, calcium, strontium, barium, chromium, manganese, iron, cobalt, nickel, copper, silver, gold, platinum, palladium, zinc, cadmium, mercury, aluminium, gallium, indium, tin, lead and bismuth. Particularly preferred is nickel.

More particularly, suitable cations include alkali metal ions (e.g. Li⁺ lithium ion, Na⁺ sodium ion, K⁺ potassium ion, Rb⁺ rubidium ion, Cs⁺ caesium ion), alkaline earth metal ions (e.g. Be²⁺ beryllium ion, Mg²⁺ magnesium ion, Ca²⁺ calcium ion, Sr²⁺ strontium ion, Ba²⁺ barium ion), transition metal ions (e.g. Ti²⁺ titanium (II) ion, Ti⁴⁺ titanium (IV) ion, V²⁺ vanadium (II) ion, V³⁺ vanadium (III) ion, V⁴⁺ vanadium (IV) ion, V⁵⁺ vanadium (V) ion, Cr²⁺ chromium (II) ion, Cr³⁺ chromium (III) ion, Cr⁶⁺ chromium (VI) ion, Mn²⁺ manganese (II) ion, Mn³⁺ manganese (III) ion, Mn⁴⁺ manganese (IV) ion, Fe²⁺ iron (II) ion, Fe³⁺ iron (III) ion, Co²⁺ cobalt (II) ion, Co³⁺ cobalt (III) ion, Ni²⁺ nickel (II) ion, Ni³⁺ nickel (III) ion, Cu⁺ copper (I) ion, Cu²⁺ copper (II) ion, Ag⁺ silver ion, Au⁺ gold (I) ion, Au³⁺ gold (III) ion, Pt²⁺ platinum (II) ion, Pt⁴⁺ platinum (IV) ion, Pd²⁺ palladium (II) ion, Pd⁴⁺ palladium (IV) ion, Zn²⁺ zinc ion, Cd²⁺ cadmium ion, Hg⁺ mercury (I) ion, Hg²⁺ mercury (II) ion), Group III metal ions (e.g. Al³⁺ aluminium ion, Ga³⁺ gallium ion, In⁺ indium (I) ion, In³⁺ indium (III) ion), Group IV metal ions (e.g. Sn²⁺ tin (II) ion, Sn⁴⁺ tin (IV) ion, Pb²⁺ lead (II) ion, Pb⁴⁺ lead (IV) ion), and/or Group V metal ions (e.g. Bi³⁺ bismuth (III) ion, Bi⁵⁺ bismuth (V) ion). Ni²⁺ (II) ion is particularly preferred.

The metal moiety may be in the form of a metal salt. Suitable metal salts include but are not limited to halides, nitriles, hydroxides and the like.

The metal moiety may be in the form of an oxide or nanoparticle. For example, iron oxide nanoparticles may be used. Other suitable oxides or nanoparticles include iron oxides, iron nitrides, iron carbides, iron metal particles, nickel oxides, nickel carbides, nickel particles, titanium oxides, titanium metal particles, titanium nitrides, titanium carbides, silver metal particles and gold metal particles.

As used herein, the term “cleaving trigger” may refer to a thermal trigger (e.g., at a temperature of above about 50° C., such as about 50° C. to about 200° C., about 60° C. to about 200° C., about 70° C. to about 200° C., about 80° C. to about 200° C., about 90° C. to about 200° C., about 100° C. to about 200° C., about 110° C. to about 200° C., about 120° C. to about 200° C., about 130° C. to about 200° C., about 140° C. to about 200° C., about 150° C. to about 200° C., about 160° C. to about 200° C., about 170° C. to about 200° C., about 180° C. to about 200° C. or about 190° C. to about 200° C.), a light trigger (e.g., UV light, visible light, or infrared light), or a (bio)chemical trigger (e.g., a solvent wash, such as with formamide, dimethylformamide, dimethylacetamide, N-methylpyrrolidinone, tetrahydrofuran, dioxane, chloroform, acetone, acetonitrile, dimethyl sulfoxide, alcohols (e.g. methanol, ethanol, isopropanol), water, acetic acid, formic acid, pyridine; acid/base treatment; a complexation agent, such as imidazole; a chelating agent, such as ethylenediamine, EDTA, ethylene glycol, glycerol; a catalyst, such as a metathesis catalyst; an oxidising or reducing agent; or an enzyme) that causes the target-binding oligonucleotides to detach from the solid support. The cleaving trigger may involve one or more of the thermal trigger, light trigger and/or (bio)chemical trigger, for example a hot solvent wash (e.g., hot formamide).

In some embodiments, it may be advantageous to use linking groups that comprise a first linking group and a second linking group on the solid support surface, wherein:
1a) a first linking group is configured to enable a first target-binding oligonucleotide to attach to said solid support via the first linking group;
1b) a second linking group is configured to enable a second target-binding oligonucleotide to attach to said solid support via the second linking group; thereafter
2a) the first linking group is configured to release the first target-binding oligonucleotide on exposure to a first cleaving trigger;
2b) the second linking group is configured to release the second target-binding oligonucleotide on exposure to a second cleaving trigger; and thereafter
3a) the first linking group is configured to enable a new first target-binding oligonucleotide to attach to the solid support via the first linking group; and
3b) the second linking group is configured to enable a new second target-binding oligonucleotide to attach to the solid support via the second linking group.

The second linking group may be different from the first linking group.

For example, a flow cell may comprise a first area comprising a first linking group, and a second area comprising a second linking group. In embodiments where the flow cell comprises nanowells, at least one of the nanowells (e.g., each of the nanowells) may be divided into two distinct areas, one corresponding with the first area, the other corresponding with the second area. For example, the at least one of the nanowells may be divided into two halves, one corresponding with the first area, the other corresponding with the second area.

The first linking group and the second linking group may be “orthogonal” to each other. For example, the term “orthogonal” used in this context may refer to when the first target-binding oligonucleotide (e.g., a P5 primer) attached to the first linking group is not (substantially) releasable on exposure to the second cleaving trigger. Alternatively, or in addition, the term “orthogonal” used in this context may refer to when the second target-binding oligonucleotide (e.g., a P7 primer) attached to the second linking group is not (substantially) releasable on exposure to the first cleaving trigger. Therefore, it is possible to selectively detach (and replace) certain first or second target-binding oligonucleotides, such as first and second primers, from the solid support in the presence of other target-binding oligonucleotides, such as other primers.

For example, the first linking group may comprise a biotin group (attachable to a first target-binding oligonucleotide comprising an avidin, e.g. streptavidin; or attachable to a first target-binding oligonucleotide comprising a biotin via an avidin bridging intermediary, e.g., streptavidin) or an avidin such as streptavidin (attachable to a first target-binding oligonucleotide comprising a biotin moiety); while the second linking group may comprise nickel (attachable to a second target-binding oligonucleotide comprising histidine, e.g. His6 tag) or a histidine moiety (attachable to a second target-binding oligonucleotide comprising nickel). The first target-binding oligonucleotide attached to the first linking group may be detached using a solvent wash (e.g., hot formamide), while the second linking group remains attached to a second target-binding oligonucleotide. The first linking group may then be reattached with a new first target-binding oligonucleotide. The second target-binding oligonucleotide attached to the second linking group may be detached using a complexation agent (e.g., imidazole), while the first linking group remains attached to a first target-binding oligonucleotide. The second linking group may then be reattached with a new second target-binding oligonucleotide. As discussed above, the first target-binding oligonucleotide may be a target capture probe or a primer. The second target-binding oligonucleotide may also be (the same or a different) target capture probe or a primer.

The first linking group may be attached to the first target-binding oligonucleotide (e.g., P5 primer). The second linking group may be attached to the second target-binding oligonucleotide (e.g., P7 primer).

The flow cell may be configured to allow multiple rounds of release and reattachment of new target-binding oligonucleotides.

In an embodiment, a flow cell may have a run to run (e.g., between an initial run and a run immediately after the initial run) DNA contamination level of less than 5%. For example, the DNA contamination level may be less than 3%, less than 2%, less than 1%, less than 0.5%, less than 0.3%, less than 0.2%, less than 0.1%, less than 0.05%, less than 0.02%, or less than 0.01%.

In an embodiment, a method of manufacturing a reusable flow cell as described herein is provided.

In some embodiments, the linking groups may comprise a biotin moiety and the target-binding oligonucleotides may comprise a biotin moiety. The target-binding oligonucleotides may be pre-incubated with an avidin (e.g., streptavidin) before the step of attaching the target-binding oligonucleotides to the linking group.

In an embodiment, a method of regenerating a reusable flow cell is provided, the method comprising:
providing a reusable flow cell as described herein; wherein the linking groups are attached to first target-binding oligonucleotides;
exposing the reusable flow cell to the cleaving trigger to remove first target-binding oligonucleotide from the linking groups;
attaching second target-binding oligonucleotides to the linking groups.

The cleaving trigger may be a thermal trigger, a light trigger or a chemical/biochemical trigger, as described herein.

In embodiments, the cleaving trigger is a solvent wash. In some embodiments, the cleaving trigger is a hot formamide wash.

In embodiments, the cleaving trigger is a complexation agent. In some embodiments, the cleaving trigger is imidazole.

In some embodiments, the method further comprises a step of treating the flow cell with a nuclease or the like (e.g., a deoxyribonuclease, such as DNase I or DNase II) prior to the step of attaching the second target-binding oligonucleotides to the linking groups. In embodiments, the nuclease is a deoxyribonuclease. In some embodiments, the nuclease is DNase I.

In some embodiments where the reusable flow cell has a linking group that comprises a first linking group and a second linking group, the step of exposing the reusable flow cell to the cleaving trigger comprises exposure to a first cleaving trigger to remove the first target-binding oligonucleotides from the first linking group, without (substantially) removing the second target-binding oligonucleotides from the second linking group. Alternatively, or in addition, the step of exposing the reusable flow cell to the cleaving trigger may comprise or further comprise exposure to a second cleaving trigger to remove the second target-binding oligonucleotides from the second linking group, without (substantially) removing the first target-binding oligonucleotides from the first linking group.

The method may then involve a step of attaching a new first target-binding oligonucleotides to the first linking group after removal of the first target-binding oligonucleotides from the first linking group. In some embodiments, the step of attaching another first target-binding oligonucleotide to the first linking group may be conducted immediately after the step of removing the first target-binding oligonucleotides from the first linking group. In other embodiments, the flow cell may be further treated with a nuclease (e.g. a deoxyribonuclease, such as DNase I or DNase II) prior to the step of removing the first target-binding oligonucleotides from the first linking group.

The method may also involve a step of attaching a new second target-binding oligonucleotides to the second linking group after removal of the second target-binding oligonucleotides from the second linking group. In some embodiments, the step of attaching another second target-binding oligonucleotide to the second linking group may be conducted immediately after the step of removing the second target-binding oligonucleotides from the second linking group. In other embodiments, the flow cell may be further treated with a nuclease (e.g., a deoxyribonuclease, such as DNase I or DNase II) prior to the step of removing the second target-binding oligonucleotides from the second linking group.

In some embodiments, the first target-binding oligonucleotides may be removed then a new first target-binding oligonucleotide reattached, followed by the second target-binding oligonucleotide being removed then a new second target-binding oligonucleotide reattached. In this way, the new first target-binding oligonucleotide and the new second target-binding oligonucleotide are selectively attached to respective first and second linking groups on the solid support.

Alternatively, both the first target-binding oligonucleotide and the second target-binding oligonucleotide may be removed, and then another first target-binding oligonucleotide and another second target-binding oligonucleotide are reattached to the solid support. Selective attachment of the another first target-binding oligonucleotide and another second target-binding oligonucleotide to respective first and second linking groups on the solid support may be conducted using reaction conditions (e.g., time, temperature, catalyst choice, pH) that permit the another first target-binding oligonucleotide to reattach to the first linking group, but not (substantially) reattach to the second linking group, and vice versa for the another second target-binding oligonucleotide.

In some embodiments, the method may comprise multiple rounds of release and reattachment of new target-binding oligonucleotides.

The present subject matter will now be described by way of the following non-limiting examples.

### EXAMPLES

### Example 1: Streptavidin-Biotin P5/P7 Surfaces

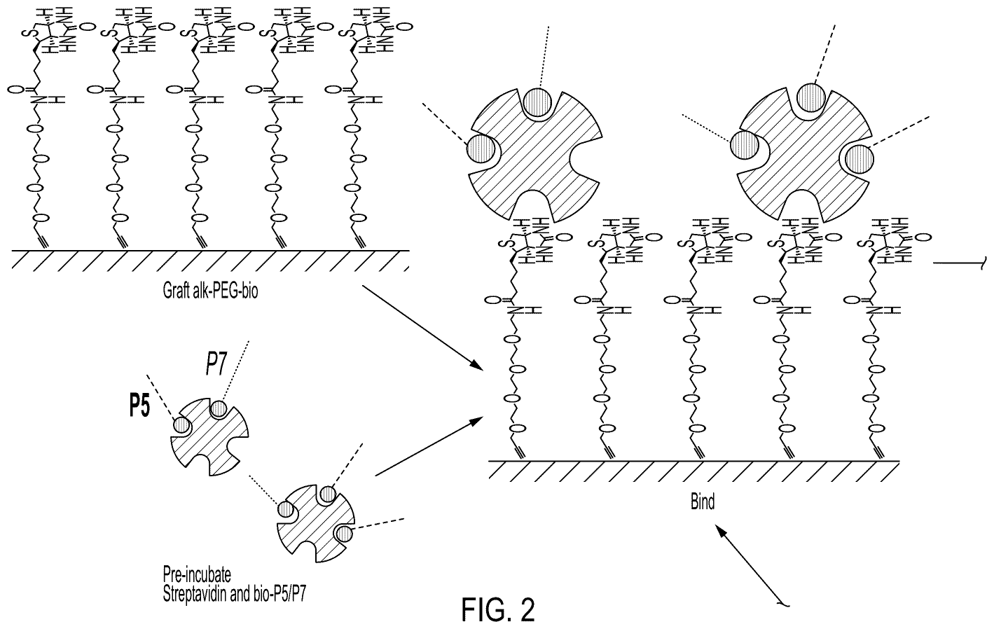

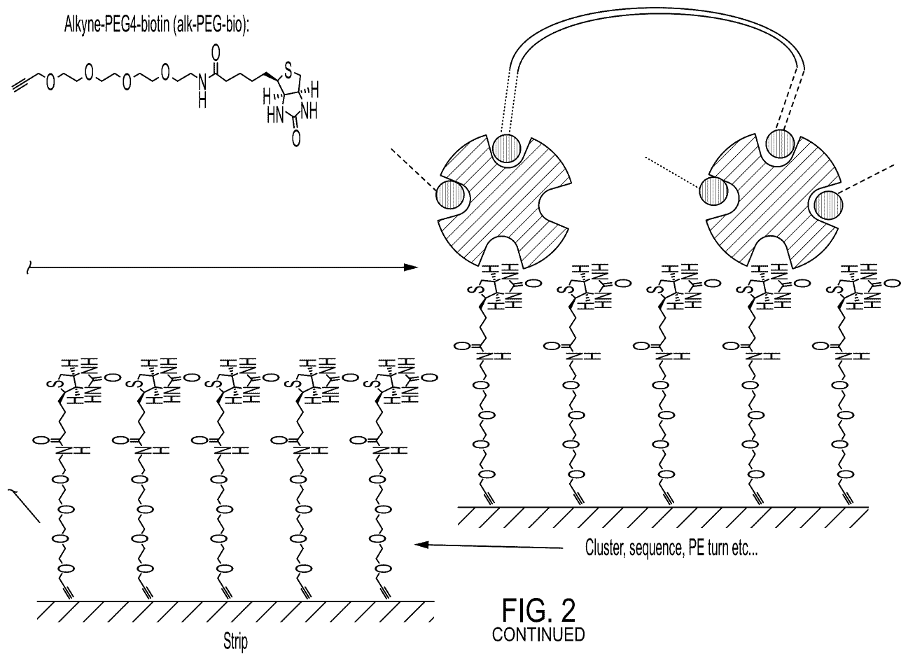

Proof-of-concept for reusable flow cells: Alkyne-PEG4-biotin is grafted to a standard poly(N-(5-azidoacetamidylpentyl)acrylamide-co-acrylamide) (PAZAM) coated and polished HiSeqX flow cell (available from Illumina, Inc). Streptavidin is pre-incubated with stoichiometric amounts of 5′ biotinylated P5 and P7 oligos. The streptavidin-bioP5/P7 complexes are then flushed over the flow cell to couple the oligos to the flow cell by the free-binding sites in the streptavidin binding to the surface grafted PEG-biotin (FIG. 2).

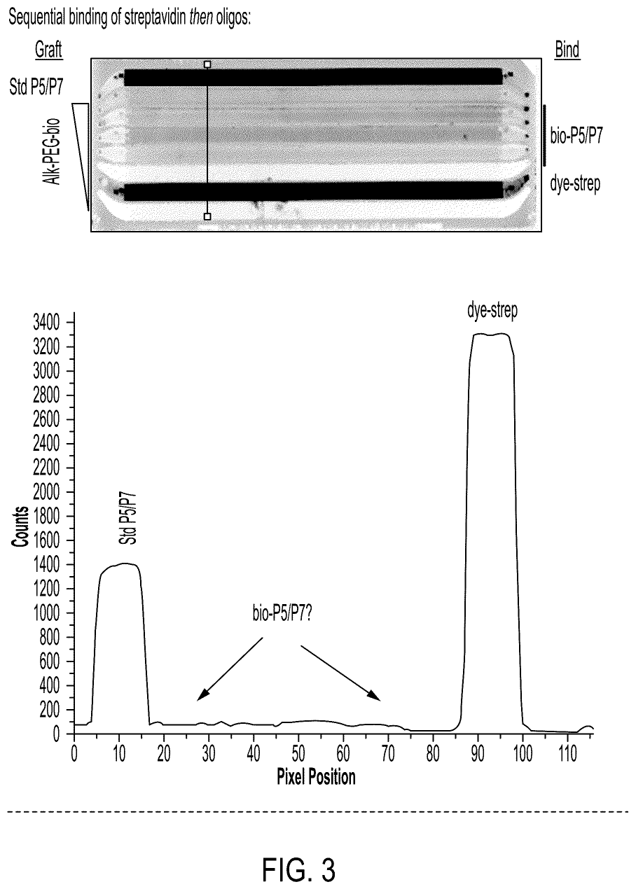

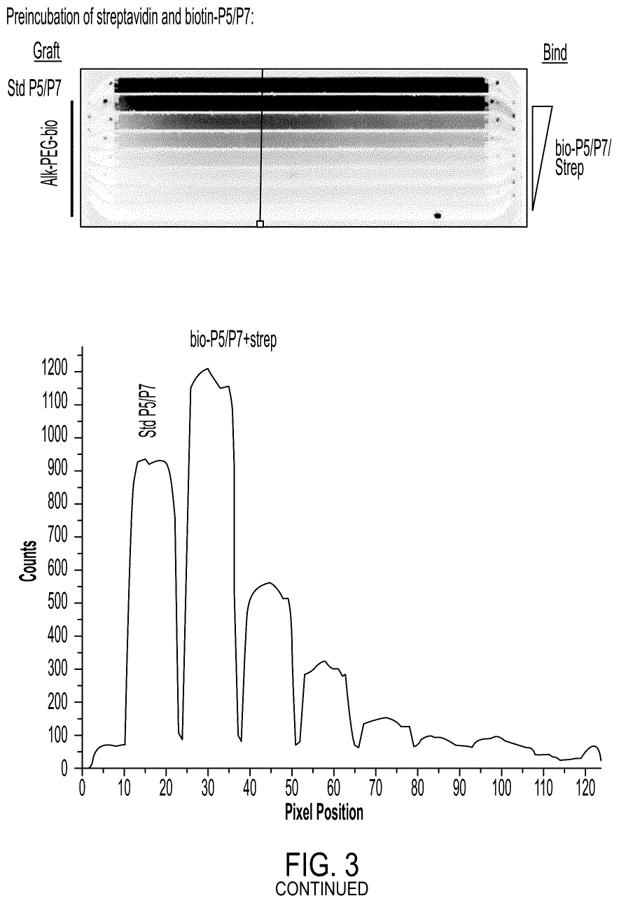

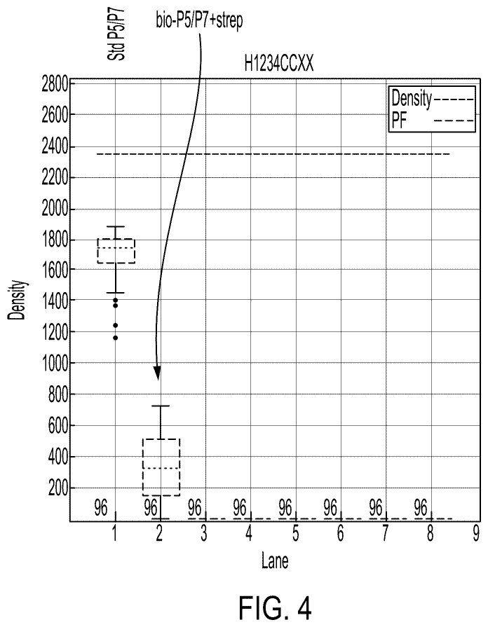

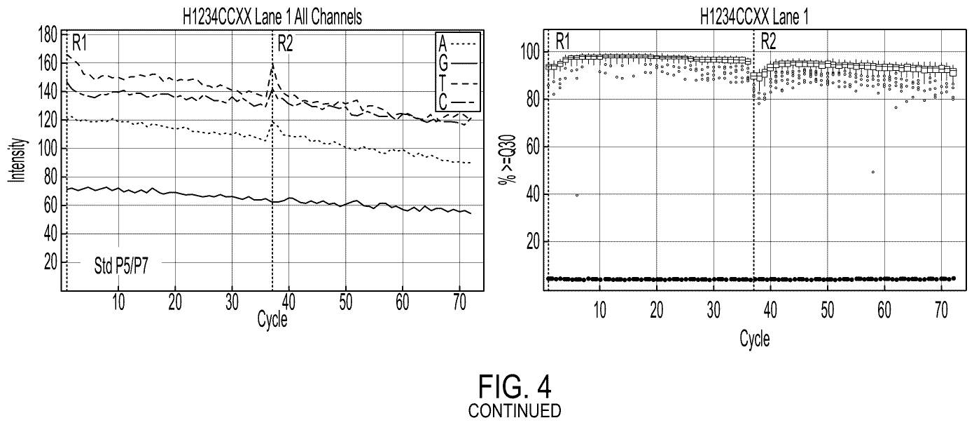

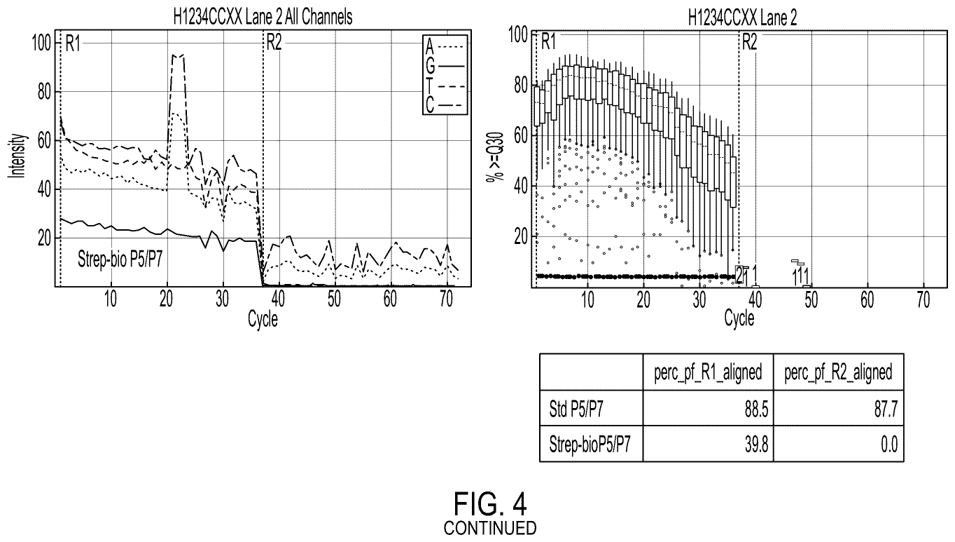

The P5/P7 oligos are shown to support cluster amplification and sequencing (FIG. 3). During the initial sequencing run, hot formamide washes were used to strip the streptavidin from the surface (FIG. 4, intensity after R2).

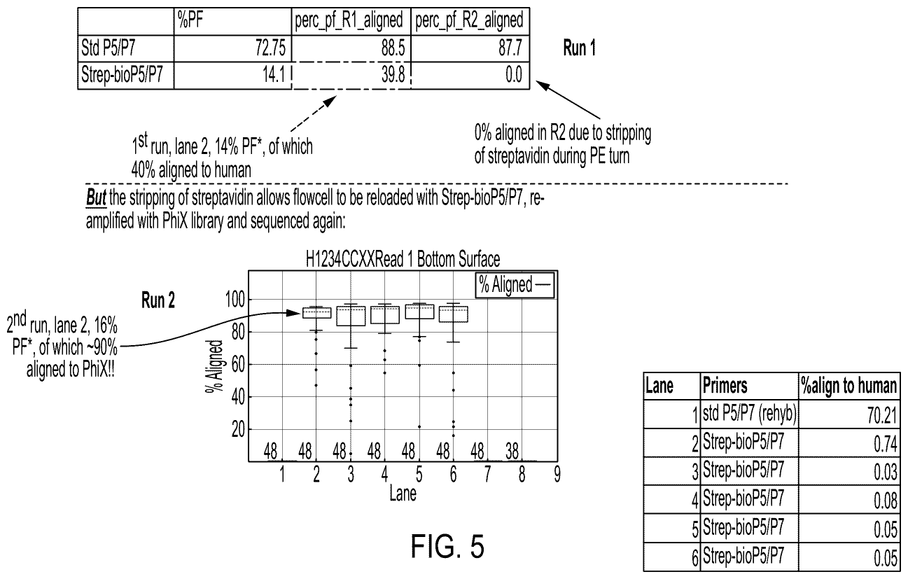

This then enabled a further round of streptavidin-oligo complex binding, followed by amplification and sequencing of a different library on the same lane of a flow cell, hence generating proof-of-concept for re-using flow cells for sequencing (FIG. 5).

### Example 2: Treatment of Streptavidin-Biotin P5/P7 Surfaces with DNase

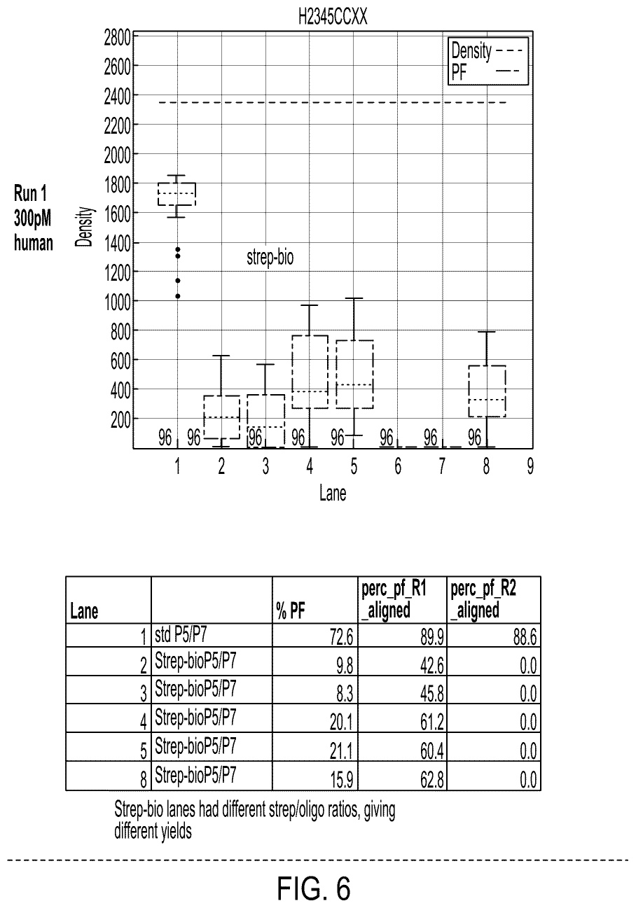

Additional reductions in run-to-run contamination: A flow cell was prepared as in Example 1 and used to sequence a human library (FIG. 6). This run yielded up to 20% PF with 60% aligning to human for the Strep-bio lanes. Then before the re-binding of the Strep-bioP5/P7 surface, some of the lanes of the flow cell were treated with DNaseI to digest away any remaining cluster strands.

The subsequent sequencing of a PhiX library on this flow cell yielded up to 53% PF, and also showed that DNase treated lanes had <0.01% carryover of clusters from the human run. Lanes which had not been DNase treated showed 1-3.4% carryover contamination.

### Example 3: Flow Cell Enrichment

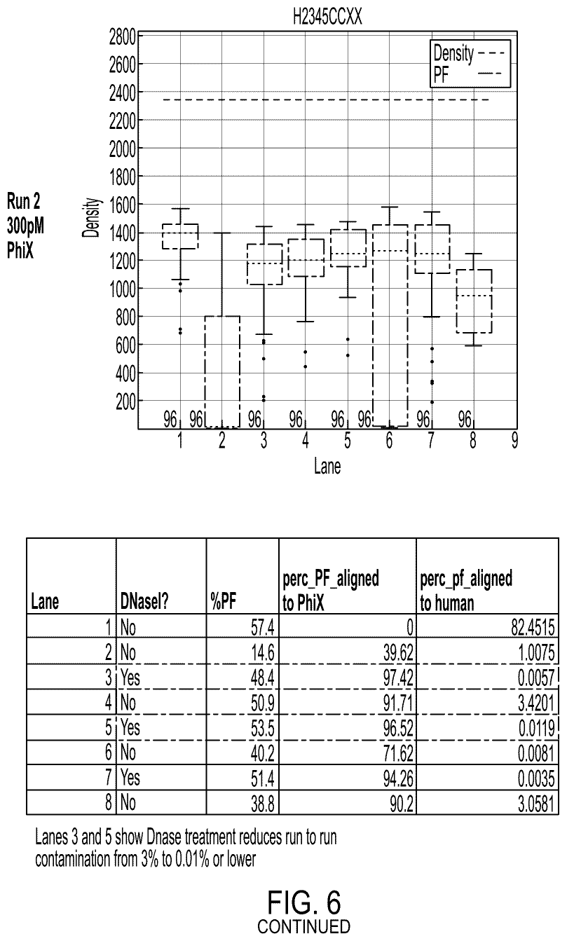

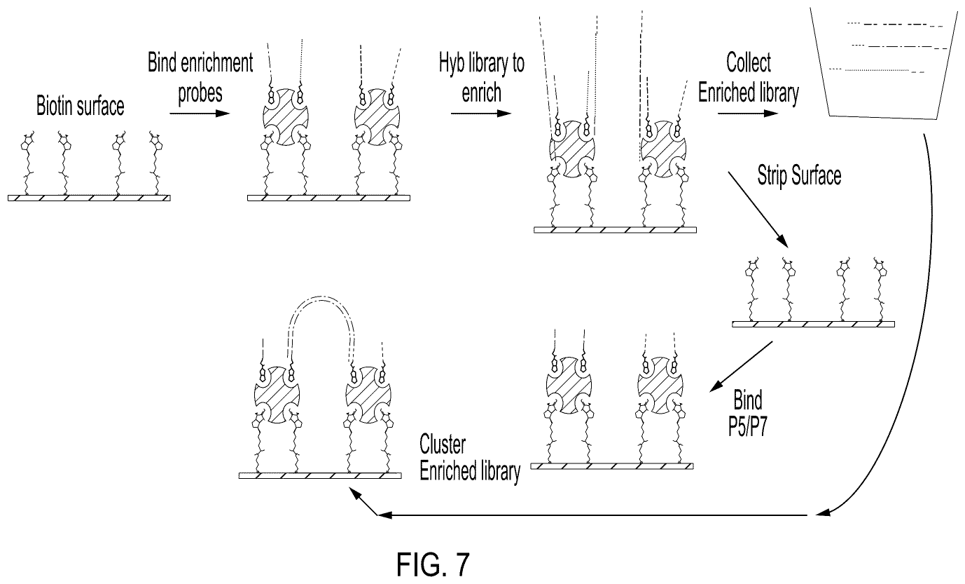

This is an example of using a reusable flow cell provided herein to perform at least one round of library enrichment prior to sequencing. Again, Alkyne-PEG4-biotin is grafted to a standard poly(N-(5-azidoacetamidylpentyl)acrylamide-co-acrylamide) (PAZAM) coated and polished HiSeqX flow cell. Streptavidin is pre-incubated with stoichiometric amounts of 5′ biotinylated target capture probes that are complementary or substantially complementary to a or a portion of the target sequence. One or more different target capture probes may be used. As shown in FIG. 7, four different target capture probes may be used. By different is meant that the target capture probes differ in nucleic acid sequence—that is, they are not 100% identical in sequence. The four different target capture probes may be used to capture different parts of the same target sequence (i.e. they bind to different regions of one target sequence). Alternatively, the four different target capture probes may be used to capture different target sequences.

The streptavidin-bio-target capture probe complexes are then flushed over the flow cell to couple the oligos to the flow cell by the free binding sites in the streptavidin binding to the surface grafted PEG-biotin (FIG. 7).

The captured target library strands are then eluted from the solid support and optionally collected in a collection well.

The solid support may then be stripped of the streptavidin-bio-target capture probe complexes—for example using hot formamide washes, allowing subsequent binding of streptavidin-bio primer (e.g. P5/P7) complexes (formed as above by a pre-incubation step using stoichiometric amounts of Streptavidin and 5′ biotinylated primers—e.g., P5 and P7 oligos).

In the final step, the collected library is flushed back over the flow cell allowing cluster amplification and sequencing of the target enriched library.

### Example 4: Multiple Rounds of Flow Cell Enrichment

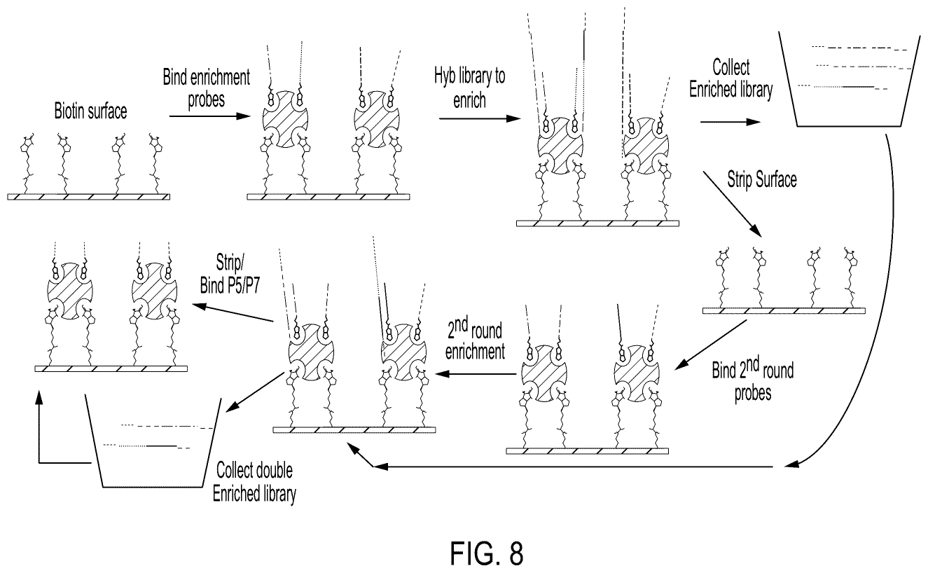

This is an example of using a reusable flow cell provided herein to perform at least two rounds of library enrichment prior to sequencing. Again, Alkyne-PEG4-biotin is grafted to a standard poly(N-(5-azidoacetamidylpentyl)acrylamide-co-acrylamide) (PAZAM) coated and polished HiSeqX flow cell. Streptavidin is pre-incubated with stoichiometric amounts of 5′ biotinylated target capture probes that are complementary or substantially complementary to a or a portion of the target sequence. One or more different target capture probes may be used. As shown in FIG. 8, four different target capture probes may be used. By different is meant that the target capture probes differ in nucleic acid sequence—that is, they are not 100% identical in sequence. The four different target capture probes may be used to capture different parts of the same target sequence (i.e. they bind to different regions of one target sequence). Alternatively, the four different target capture probes may be used to capture different target sequences.

The streptavidin-bio-target capture probe complexes are then flushed over the flow cell to couple the oligos to the flow cell by the free-binding sites in the streptavidin binding to the surface grafted PEG-biotin (FIG. 8).

The captured target library strands are then eluted from the solid support and optionally collected in a collection well.

The solid support may then be stripped of the streptavidin-bio-target capture probe complexes—for example using hot formamide washes—and another round of target capture can begin by flushing over a second set of streptavidin-bio-target capture probe complexes, which subsequently bind to the free binding sites in the surface grafted PEG-biotin (FIG. 8) followed by flushing over the collected library. A second or further (e.g., third or so on) round of target capture before sequencing can further improve specificity of the target capture process. The second (and/or further) set of target capture probes may bind to the same or different sequences within the target sequence.

In a final step, the captured target library strands are eluted from the solid support, and optionally collected in a collection well, and the solid support is stripped of the streptavidin-bio-target capture probe complexes—for example using hot formamide washes. Next, streptavidin-bio primer (e.g., P5/P7) complexes (formed as above by a pre-incubation step using stoichiometric amounts of Streptavidin and 5′ biotinylated primers—e.g., P5 and P7 oligos) are flushed over the solid support and allowed to bind the surface grafted PEG-biotin (FIG. 8). The collected library is then flushed back over the flow cell allowing cluster amplification and sequencing of the target enriched library.

### Example 5: Localization of Enzyme Complex on Flow Cell

This is an example of using a target-binding oligonucleotide to localise an enzyme or enzyme complex to relevant site(s) on the flow cell, thereby adding further functionality to the reusable flow cell provided herein. As described above, a target-binding oligonucleotide may bind (e.g., hybridise) to an enzyme or enzyme complex to localise said enzyme or enzyme complex to a given area on the flow cell. In a similar, but alternative arrangement, a target-binding oligonucleotide may comprise (i.e., may be a part of) an enzyme or enzyme complex directly (e.g., may be attached covalently or via a sequence).

As shown in FIG. 9, Alkyne-PEG4-biotin is grafted to a standard poly(N-(5-azidoacetamidylpentyl)acrylamide-co-acrylamide) (PAZAM) coated and polished HiSeqX flow cell. Streptavidin is pre-incubated with stoichiometric amounts of 5′ biotinylated first target-binding oligonucleotide that are complementary or substantially complementary to a or a portion of the target sequence. As described above, the 5′ biotinylated first target-binding oligonucleotide may be a transposome comprising an enzyme or enzyme complex, or may include adapter sequences that may bind to an enzyme or enzyme complex in an additional step. As shown in FIG. 9, the 5′ biotinylated target-binding oligonucleotide is bound to an enzyme. More than one different target-binding oligonucleotide may be used across a flow cell (FIG. 9). By different is meant that the target-binding oligonucleotide differ in nucleic acid sequence—that is, they are not 100% identical in sequence. The different target-binding oligonucleotides may be used to bind different parts of the same target sequence (i.e., they bind to different regions of one target sequence). Alternatively, the different target-binding oligonucleotides may be used to bind different target sequences.

In FIG. 9, the enzyme complex is a transposome that comprises a sequence that is complementary or substantially complementary to a or a portion of the target sequence. In this way, streptavidin-bio-target-binding oligonucleotide-transposome complexes are formed.

In this example, the streptavidin-bio-target-binding oligonucleotide-transposome complexes are then flushed over the flow cell and couple the oligos to the flow cell by the free-binding sites in the streptavidin binding to the surface grafted PEG-biotin (FIG. 9).

By arranging the streptavidin-bio-target-binding oligonucleotide-transposome complexes across a flow cell, or within each nanowell, a library strand may be bound between different complexes. Thus, a duplex nucleotide sequence may be captured by binding to the target-recognition sequence of two separate transposomes. The bound sequences are fragmented by the enzyme, thereby creating a population of fragmented nucleic acid molecules. In particular, a library strand may be bound to a first transposon at the 5′ end of the strand and bound to a second transposon at the 3′ end of the strand. Further, by configuring the distribution of streptavidin-bio-target capture probe-transposome complexes it is possible to capture a library strand of a desired insert length, thereby increasing the efficiency of subsequent sequencing.

Due to the properties of a transposome, the captured nucleotide will be tagmented during the capture stage. Additionally, in the same or subsequent round of capture, a transposome complex may be used to index the library strands. As described above, fragmentation results in simultaneous fragmentation of the DNA and ligation of adapters to the 5′ ends of both strands of the duplex. Therefore, in some embodiments, dual-indexed paired-end libraries may be prepared from a DNA sample using a combined tagmentation and indexing step. This combined tagmentation and indexing reduces the time required for the workflow, by omitting certain wash steps and denaturation steps.

The captured tagmented target library strands are then eluted from the solid support and optionally collected in a collection well.

The solid support may then be stripped of the streptavidin-bio-target-binding oligonucleotide complexes—for example using hot formamide washes—and a round of target enrichment can begin by flushing over a set of streptavidin-bio-target capture probe complexes, which subsequently bind to the free-binding sites in the surface grafted PEG-biotin (FIG. 9) followed by flushing over the collected library. A second or further (e.g., third or so on) round of target capture before sequencing can further improve specificity of the target capture process. The second (or further) set of target capture probes may bind to the same or different sequences within the target sequence. Any such further rounds of target capture may or may not utilise an enzyme or enzyme complex. As shown in FIG. 9, in one embodiment, a transposome complex is used only in the first round of target capture, and the second round of capture is facilitated by second target-binding oligonucleotides comprising primers.

SEQUENCE LISTING
SEQ ID NO: 1: P5 sequence
AATGATACGGCGACCACCGAGATCTACAC
SEQ ID NO: 2: P7 sequence
CAAGCAGAAGACGGCATACGAGAT
SEQ ID NO: 3 P5′ sequence
(complementary to P5)
GTGTAGATCTCGGTGGTCGCCGTATCATT
SEQ ID NO: 4 P7′ sequence
(complementary to P7)
ATCTCGTATGCCGTCTTCTGCTTG
SEQ ID NO: 5 A14 sequence
TCGTCGGCAGCGTC
SEQ ID NO: 6 B15 sequence
GTCTCGTGGGCTCGG
SEQ ID NO: 7 ME sequence
AGATGTGTATAAGAGACAG
SEQ ID NO: 8 ME′ sequence
5′-phos-CTGTCTCTTATACACATCT
SEQ ID NO: 9 A14-ME
TCGTCGGCAGCGTCAGATGTGTATAAGAGACAG
SEQ ID NO: 10 B15-ME:
GTCTCGTGGGCTCGGAGATGTGTATAAGAGACAG
SEQ ID NO: 11: Alternative P5 sequence
AATGATACGGCGACCGA
SEQ ID NO: 12: Alternative P5′ sequence
(complementary to alternative P5 sequence)
TCGGTCGCCGTATCATT

1. A kit comprising:
a. a reusable flow cell, comprising a solid support and linking groups on the solid support surface; wherein the linking groups are configured to:
i. enable first target-binding oligonucleotides to attach to the solid support via the linking groups; and thereafter
ii. release the first target-binding oligonucleotides on exposure to a cleaving trigger; and thereafter
iii. enable second target-binding oligonucleotides to attach to the solid support via the linking groups; and
b. first and second target-binding oligonucleotides.

2-10. (canceled)

11. A reusable flow cell, comprising:
a solid support;
linking groups on the surface of the solid support; and
first target-binding oligonucleotides,
wherein the first target-binding oligonucleotides are attached to the solid support via the linking groups; and
wherein the linking groups are further configured to:
a. release the first target-binding oligonucleotides on exposure to a cleaving trigger; and thereafter
b. enable second target-binding oligonucleotides to attach to the solid support via the linking groups.

12. The reusable flow cell of claim 11, wherein the first target-binding oligonucleotides comprise primers.

13. The reusable flow cell of claim 11, wherein the first target-binding oligonucleotides comprise target capture probes.

14. The reusable flow cell of claim 11, wherein the second target-binding oligonucleotides comprise primers.

15. The reusable flow cell of claim 11, wherein the first and second target-binding oligonucleotides each comprise one or more linking groups configured to interact with the linking groups on the solid support surface.

16. The reusable flow cell of claim 11, wherein the linking groups are configured to form non-covalent interactions with the first and second target-binding oligonucleotides.

17. The reusable flow cell of claim 16, wherein the linking groups comprise a biotin moiety or an avidin.

18. The reusable flow cell of claim 11, wherein the linking groups are configured to form reversible covalent bonds with the first and second target-binding oligonucleotides.

19. The reusable flow cell of claim 11, wherein the linking groups are configured to form metal-coordination bonds with the first and second target-binding oligonucleotides.

20. The reusable flow cell of claim 19, wherein the linking groups comprise nickel or histidine.

21. The reusable flow cell of claim 11, wherein the linking groups comprise first and second linking groups on the solid support surface; wherein:
a) the first linking group is configured to enable first target-binding oligonucleotides to attach to said solid support via the first linking group;
b) the second linking group is configured to enable second target-binding oligonucleotides to attach to said solid support via the second linking group;
2a) the first linking group is configured to release the first target-binding oligonucleotides on exposure to a first cleaving trigger;
2b) the second linking group is configured to release the second target-binding oligonucleotides on exposure to a second cleaving trigger;
3a) the first linking group is configured to enable new first target-binding oligonucleotides to attach to the solid support via the first linking group; and
3b) the second linking group is configured to enable new second target-binding oligonucleotides to attach to the solid support via the second linking group.

22. The reusable flow cell of claim 21, wherein the first linking group is orthogonal to the second linking group.

23. The reusable flow cell of claim 21, wherein the linking groups are attached to the target-binding oligonucleotides.

24. The reusable flow cell of claim 21, wherein the first linking group is attached to the first target-binding oligonucleotides.

25. The reusable flow cell of claim 21, wherein the second linking group is attached to the second target-binding oligonucleotides.

26-29. (canceled)

30. Use of the reusable flow cell according to claim 11 in sequencing, cluster counting or target enrichment.

31. (canceled)

32. A method of manufacturing the reusable flow cell of claim 11, said method comprising attaching the linking groups to the flow cell.

33. (canceled)

34. A method of regenerating reusable flow cell of claim 11, the method comprising:
exposing the reusable flow cell to the cleaving trigger to remove the first target-binding oligonucleotides from the linking groups; and thereafter
attaching second target-binding oligonucleotides to the linking groups.

35-39. (canceled)

40. A method of regenerating the reusable flow cell of claim 21, the method comprising:
exposing the reusable flow cell to a first cleaving trigger to remove the first target-binding oligonucleotide from the first linking group, without removing the second target-binding oligonucleotide from the second linking group.

41-43. (canceled)


SEQUENCE LISTING (12 sequences)

SEQ ID NO: 1 - DNA, 29 bp, synthetic construct
  source 1..29: mol_type = other DNA; note = P5 sequence
aatgatacggcgaccaccgagatctacac

SEQ ID NO: 2 - DNA, 24 bp, synthetic construct
  source 1..24: mol_type = other DNA; note = P7 sequence
caagcagaagacggcatacgagat

SEQ ID NO: 3 - DNA, 29 bp, synthetic construct
  source 1..29: mol_type = other DNA; note = complementary to P5
gtgtagatctcggtggtcgccgtatcatt

SEQ ID NO: 4 - DNA, 24 bp, synthetic construct
  source 1..24: mol_type = other DNA; note = complementary to P7
atctcgtatgccgtcttctgcttg

SEQ ID NO: 5 - DNA, 14 bp, synthetic construct
  source 1..14: mol_type = other DNA; note = A14 sequence
tcgtcggcagcgtc

SEQ ID NO: 6 - DNA, 15 bp, synthetic construct
  source 1..15: mol_type = other DNA; note = B15 sequence
gtctcgtgggctcgg

SEQ ID NO: 7 - DNA, 19 bp, synthetic construct
  source 1..19: mol_type = other DNA; note = ME sequence
agatgtgtataagagacag

SEQ ID NO: 8 - DNA, 19 bp, synthetic construct
  source 1..19: mol_type = other DNA; note = MEprime sequence
ctgtctcttatacacatct

SEQ ID NO: 9 - DNA, 33 bp, synthetic construct
  source 1..33: mol_type = other DNA; note = A14-ME
tcgtcggcagcgtcagatgtgtataagagacag

SEQ ID NO: 10 - DNA, 34 bp, synthetic construct
  source 1..34: mol_type = other DNA; note = B15-ME
gtctcgtgggctcggagatgtgtataagagacag

SEQ ID NO: 11 - DNA, 17 bp, synthetic construct
  source 1..17: mol_type = other DNA; note = Alternative P5 sequence
aatgatacggcgaccga

SEQ ID NO: 12 - DNA, 17 bp, synthetic construct
  source 1..17: mol_type = other DNA; note = complementary to alternative P5 sequence
tcggtcgccgtatcatt